(19) United States
(12) Patent Application Publication
(10) Pub. No.: US 2017/0050044 A1
(43) Pub. Date: Feb. 23, 2017

(54) Integumentary System Therapy

(71) Applicants: Russell M. Lebovitz, Oakland, CA (US); Kraig K. Anderson, San Mateo, CA (US)

(72) Inventors: Russell M. Lebovitz, Oakland, CA (US); Kraig K. Anderson, San Mateo, CA (US)

(21) Appl. No.: 15/212,275

(22) Filed: Jul. 17, 2016

Related U.S. Application Data
(60) Provisional application No. 62/193,749, filed on Jul. 17, 2015.

(51) Int. Cl. A61N 5/06; A61K 8/49; A61N 1/30; A61Q 19/02

(52) U.S. Cl. CPC A61N 5/062; A61Q 19/02; A61K 8/49; A61N 1/303; A61K 2800/83; A61K 2800/28; A61N 2005/067

(57) ABSTRACT

Described are methods, kits, apparatus, and compositions for integumentary system therapy. For example, a method for therapy may include providing a subject in need of therapy for a condition. The condition may be associated with a substrate located in the subject's integumentary system. The method may include contacting a therapeutic agent and the substrate in the subject's integumentary system. The method may include modulating a depth of at least one ionic species in the subject's integumentary system. The at least one ionic species may include one or more of: the therapeutic agent; a bound therapeutic agent:substrate complex; and a reaction product of one or both of the therapeutic agent and the substrate. The method may be effective to at least partly ameliorate the condition in the subject.

### CROSS-REFERENCE TO RELATED APPLICATIONS

This application claims priority to U.S. Provisional Patent Application No. 62/193,749, filed on Jul. 17, 2015, which is incorporated by reference herein in its entirety.

### BACKGROUND

Various conditions of the integumentary system include the presence of substances, organisms, cellular abnormalities, and the like in various skin layers, e.g., between the epidermis and the dermis, within the epidermis, between the living epidermal cells and the stratum corneum, in the nail bed, in hair follicles, and the like.

For example, areas of hyperpigmentation may include lentigo, commonly referred to as “liver spots” or “age spots.” In lentigo, excess extracellular melanin may accumulate near the interface between the epidermis and dermis, within the epidermis, and between the living epidermal cells and the stratum corneum. This excess extracellular melanin may persist between the epidermis and dermis, within the epidermis, and between the living epidermal cells and the stratum corneum and may not disappear during normal epidermal skin cell maturation and exfoliation. Lentigo spots may be cosmetically undesirable and may obscure signs of skin cancer or other pathological conditions. Various methods have been used to remove lentigo spots, such as surgery, cryotherapy, and application of caustic peeling or bleaching agents. However, these methods may be invasive, painful, and of poor efficacy. Ephelides (freckles) are areas of hyperpigmentation that may be related to lentigo, though typically ephelides darken with sun exposure to a greater extent than lentigo.

The present application appreciates that the treatment of conditions of the integumentary system may be a challenging endeavor.

### SUMMARY

In one embodiment, a method for therapy is provided. The method may include providing a subject in need of therapy for a condition. The condition may be associated with a substrate located in the subject's integumentary system. The method may include contacting a therapeutic agent and the substrate in the subject's integumentary system. The method may include modulating a depth of at least one ionic species in the subject's integumentary system. The at least one ionic species may include one or more of: the therapeutic agent; a bound therapeutic agent:substrate complex; and a reaction product of one or both of the therapeutic agent and the substrate. The method may be effective to at least partly ameliorate the condition in the subject.

In another embodiment, a kit for therapy is provided. The kit may include a therapeutic agent. The kit may also include instructions. The instructions may direct a user to at least partly ameliorate a condition associated with a substrate located in a subject's integumentary system. The instructions to the user may include providing the subject in need of therapy for the condition. The instructions to the user may include contacting the therapeutic agent and the substrate in the subject's integumentary system. The instructions to the user may include modulating a depth of at least one ionic species in the subject's integumentary system. The at least one ionic species may include one or more of: the therapeutic agent; a bound therapeutic agent:substrate complex; and a reaction product of one or both of the therapeutic agent and the substrate.

In one embodiment, an iontophoresis apparatus for therapy is provided. The iontophoresis apparatus may include a therapeutic agent. The therapeutic agent may include one or more of: a dye, a skin lightening agent, an oxidant, a reductant, and an agent that blocks synthesis or maturation of melanin.

In another embodiment, an apparatus for therapy of a subject's integumentary system is provided. The apparatus may include a therapeutic composition comprising a therapeutic agent. The therapeutic agent may include one or more of: a dye, a skin lightening agent, an oxidant, a reductant, and an agent that blocks synthesis or maturation of melanin. The apparatus may include a mobilization module configured to operatively couple one or more of energy or a permeation enhancer to the subject's integumentary system. The mobilization module may be effective to modulate a depth of at least one ionic species in the subject's integumentary system. The mobilization module may be effective to modulate the mobility of the at least one ionic species in the subject's integumentary system. The subject's integumentary system may include a substrate associated with a condition in need of therapy. The at least one ionic species may include one or more of: the therapeutic agent; a bound therapeutic agent:substrate complex; and a reaction product of one or both of the therapeutic agent and the substrate.

In one embodiment, a therapeutic composition is provided. The therapeutic composition may include a therapeutic agent. The therapeutic agent may include an ionic one or more of: a dye, a skin lightening agent, an oxidant, a reductant, and an agent that blocks synthesis or maturation of melanin. The therapeutic composition may include a permeation enhancer. The therapeutic agent and the permeation enhancer may be combined together in an isotonic solution to form the therapeutic composition.

### BRIEF DESCRIPTION OF THE DRAWINGS

The accompanying figures, which are incorporated in and constitute a part of the specification, illustrate example methods and apparatuses, and are used merely to illustrate example embodiments.

FIG. 1 is a flow diagram illustrating an example method for therapy.

FIG. 2 is a block diagram illustrating an example kit for therapy.

FIG. 3 is a block diagram illustrating an example apparatus for therapy.

FIG. 4A is a photo showing the dye impregnation in a subject's skin according to EXAMPLE 1.

FIG. 4B is a photo showing removal of impregnated dye from a subject's skin according to EXAMPLE 2.

FIG. 4C is a photo showing removal of impregnated dye from a subject's skin according to EXAMPLE 2.

FIG. 5 is a collection of three images showing aspects of dye in a subject's skin according to EXAMPLE 7.

FIG. 6 is a collection of three images showing aspects of dye in a subject's skin according to EXAMPLE 8.

FIG. 7 is an image showing aspects of dye in a subject's skin according to EXAMPLE 9.

FIG. 8 is a collection of three images showing aspects of dye in a subject's skin according to EXAMPLE 10.

FIG. 9 is a collection of two images showing aspects of dye in a subject's skin according to EXAMPLE 12.

### DETAILED DESCRIPTION

The present application generally relates to methods of therapy, e.g., for treating conditions in a subject associated with associated with a substrate located in the subject's integumentary system, e.g., pigmentation conditions associated with melanin located in the skin.

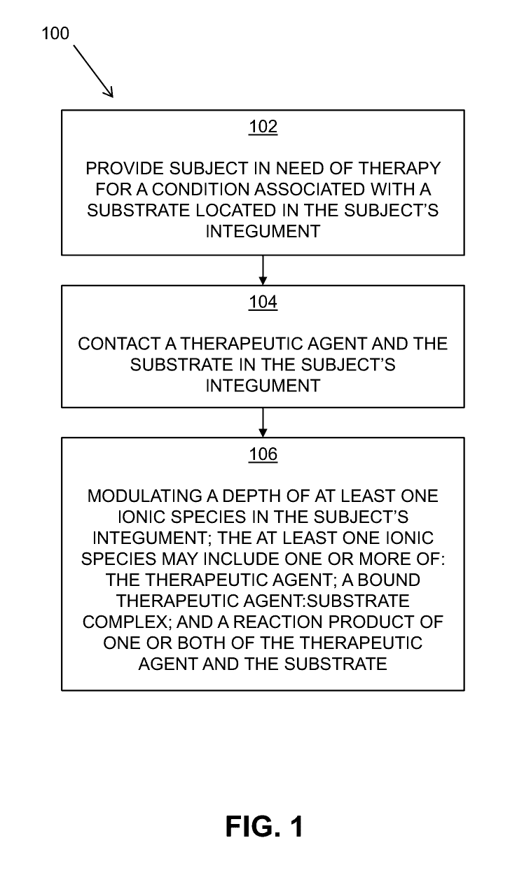

FIG. 1 is a flow diagram illustrating an example method for therapy 100. In various embodiments, method 100 may include 102 providing a subject in need of therapy for a condition. The condition may be associated with a substrate located in the subject's integumentary system. Method 100 may include 104 contacting a therapeutic agent and the substrate in the subject's integumentary system. Method 100 may include 106 modulating a depth of at least one ionic species in the subject's integumentary system. The at least one ionic species may include one or more of: the therapeutic agent; a bound therapeutic agent:substrate complex; and a reaction product of one or both of the therapeutic agent and the substrate. Method 100 may be effective to at least partly ameliorate the condition in the subject.

In some embodiments, the method may include treating the subject's integumentary system to facilitate permeation, e.g., mobility of the at least one ionic species with respect to the subject's integumentary system. For example, the method may include applying energy to the subject's integumentary system. The energy may be effective to modulate the depth of the at least one ionic species with respect to the subject's integumentary system. The energy may be effective to facilitate permeation, e.g., mobility of the at least one ionic species with respect to the subject's integumentary system. The energy may include, for example, one or more of mechanical energy, thermal energy, and electromagnetic energy. Mechanical energy may be applied, for example, through one or more of abrasion, shear, vacuum, pressure, suction, impact, pressurized flow, stirring, ultrasound, alternating tension and compression, vibration, torsion, and the like. Thermal energy may be provided, for example, by heating. Energy delivered electromagnetically may encompass energy delivered by electrical fields, magnetic fields, electromagnetic radiation, and the like, for example, energy provided by laser light, filtered light, diode light, sunlight, radiofrequency energy, electrical fields effective to cause iontophoresis, and the like.

In some embodiments, the energy may be provided by any selection from the preceding lists, or any combination thereof. For example, the energy may be provided by ultrasound, e.g., effective to cause phonophoresis. The energy may be provided by an electrical field, e.g., a direct current effective to cause iontophoresis, a pulsed current effective to cause electroporation, and the like. The energy may be provided optically, e.g., by a laser effective to cause mechanical waves resulting in photomechanical portion. The energy may be provided by heating. The energy may be provided by mechanical abrasion, which may also provide thermal energy via friction.

In various embodiments, the energy may be provided before application of the therapeutic agent, for example, a subject's skin may be pretreated with ultrasound to increase skin permeability prior to contact with the therapeutic agent. In some embodiments, the energy may be applied during application of the therapeutic agent to the integumentary system, for example, a subject's skin may be contacted with a therapeutic agent in the presence of phonophoresis, iontophoresis, or the like to drive the therapeutic agent into the skin. In several embodiments, the energy may be applied to move the ionic species within the integumentary system or to extract the ionic species from the integumentary system, for example, during or after binding or reaction of the therapeutic agent with the substrate. with ultrasound to increase skin permeability prior to contact with the therapeutic agent.

In several embodiments, the method may include contacting the subject's integumentary system with a permeation enhancer. The permeation enhancer may include any permeation enhancing agent known to the art, such as a chemical permeation enhancer or a physical permeation enhancer. The permeation enhancer may be effective to enhance permeation of the at least one ionic species, e.g., the therapeutic agent, across, within, through, or out of the subject's integumentary system. For example, a subject's skin may include a stratum corneum, which may provide a barrier to entry of the therapeutic agent.

Examples of chemical permeation enhancers may include one or more of: a sulfoxide, e.g., dimethylsulfoxide; an amide, e.g., dimethylacetamide, or dimethylformamide; a pyrrolidone, e.g., 2-pyrrolidone, N-methyl-2-pyrrolidone, or 1-lauryl-2-pyrrolidone; an alcohol, e.g., ethanol, 1-octanol, 1-hexanol, 1-decanol, lauryl alcohol, linolenyl alcohol, or glycerol; a glycol, e.g., propylene glycol, butane-1,2-diol, or polyethylene glycol 400; an ester, e.g., glyceride esters, monoolein, fatty acid esters such as cetyl lactate, butylacetate, or isopropyl myristate; a urea, e.g., urea, 1-dodecylurea, 1-dodecyl-3-methylurea, or 1-dodecyl-3-methylthiourea; a lactam, e.g., 1-alkyl-azacycloalkanones such as 1-dodecylazacycloheptan-2-one (Azone or laurocapram), or 1-alkenylazacycloalkanones; an enzyme, e.g., Acid phosphatase, calonase, or papain; an imino sulfurane, e.g., S,S-dimethyl-N-(5-nitro-2-pyridyl) imino sulfurane, or S, S-dimethyl-N-(4-bromobenzoyl) imino sulfurane; a cyclodextrin, e.g., 2-hydroxypropyl-O-cyclodextrin, or methylated-O-cyclodextrin; a fatty acid, e.g., alkanoic acids, oleic acid, lauric acid, or capric acid; an alkyl N,N di-substituted amino acetate, e.g., dodecyl 2-(N,N-dimethylamino)propionate (DDAIP); an essential oil, e.g., terpenes, terpenoids, and other oils such as cineole, eugenol, camphor, menthol, D-limonene, nerolidol, farnesol, carvone, or menthone; a polymer, e.g., β-D-glucopyranosyl-terminated oligodimethylsiloxanes, or 1-alkyl-3-β-D -glucopyranosyl-1,1,3,3-tetramethyldisiloxanes; an oxazolidinone, e.g., 4-decyloxazolidin-2-one or 3-acetyl-4-decyloxazolidin-2-one; a surfactant; combinations thereof, and the like.

Example surfactants may include a nonionic surfactant, a cationic surfactant, an anionic surfactant, a zwitterionic surfactant, and combinations thereof. Surfactants may be natural or synthetic. Nonionic surfactants may include, for example: fatty alcohols, e.g., cetyl alcohol, stearyl alcohol, or oleyl alcohol; polyoxyalkylene glycol alkyl ethers, such as polyoxyethylene glycol alkyl ethers (e.g., Brij series) or polyoxyethylene glycol alkyl ethers; glucoside alkyl ethers, e.g., decyl, lauryl, or octyl glucoside; polyoxyethylene glycol octylphenol ethers, e.g., Triton X-100; sorbitan alkyl esters, e.g., sorbitan monopalmitate, sorbitan dilaurate, sorbitan dioleate, sorbitan monolaurate, sorbitan monooleates, sorbitan trilaurate, sorbitan trioleate, Span 20, Span 40, or Span 85; polyoxyethylene sorbitan esters such as polyoxyethylene (20) sorbitan monooleates (Polysorbate 80); polyoxyethylene glycol alkylphenol ethers, e.g., nonoxynol or octoxynol; poloxamers, e.g. block copolymers of polyethylene glycol and polypropylene glycol such as pluronic and synperonic np; and the like. Cationic surfactants may include, for example, quaternary tetraalkyl or benzyltrialkyl ammonium halides such as cetyl trimethyl ammonium bromide, and the like. Anionic surfactants may include, for example, sodium dodecyl sulfate, sodium lauryl sulfate, n-lauroyl sarcosine, sodium laurate, sodium oleate, sodium phenylsulfonate, and the like. Zwitterionic surfactants may include, for example, betaines, sultaines, phosphatidylserine, phosphatidylethanolamine, phosphatidylcholine, and the like.

In various embodiments, the permeation enhancer may include two or more surfactants. For example, the permeation enhancer may include a nonionic surfactant and a zwitterionic surfactant. For example, the nonionic surfactant may include one or more of: polyethylene glycol dodecyl ether such as polyoxyethylene 4-dodecyl ether (Brij 30), polyoxyethylene 23-lauryl ether (Brij 35), polyoxyethylene 2-cetyl ether (Brij 52), polyoxyethylene 10-cetyl ether (Brij C10), polyoxyethylene 20-cetyl ether (Brij 58), polyoxyethylene 2-stearyl ether, polyoxyethylene 10-stearyl ether, polyoxyethylene 20-stearyl ether, polyoxyethylene 2-oleyl ether, polyoxyethylene 10-oleyl ether, polyoxyethylene 100-stearyl ether, and polyoxyethylene 21-stearyl ether. The zwitterionic surfactant may include one or more of: 3-(decyl dimethyl ammonio) propane sulfonate (DPS), 3-(dodecyl dimethyl ammonio) propane sulfonate (DDPS), tetradecyldimethylammonio propane sulfonate (TPS), hexadecyldimethylammonio propane sulfonate (HPS), octadecyldimethylammonio propane sulfonate (OPS), cocamidopropyl betaine, oleyl betaine, cocamidopropyl hydroxysultaine, and 3-(3-cholamidopropyl)-dimethylammonio-1-propanesulfonate. In some embodiments, the permeation enhancer may include at least two surfactants, e.g., DPS, Brij30; DPS, Brij35; DPS, Brij52; DPS, BrijC10; DPS, Brij58; DDPS, Brij30; DDPS, Brij35; DDPS, Brij52; DDPS, BrijC10; DDPS, Brij58; TPS, Brij30; TPS, Brij35; TPS, Brij52; TPS, BrijC10; TPS, Brij58; HPS, Brij30; HPS, Brij35; HPS, Brij52; HPS, BrijC10; HPS, Brij58; OPS, Brij30; OPS, Brij35; OPS, Brij52; OPS, BrijC10; OPS, Brij58. In several embodiments, the permeation enhancer may exclude one or more of the preceding surfactant pairs. In some embodiments, the permeation enhancer may include at least two surfactants, e.g., OPS, BrijC10; TPS, BrijC10; HPS, Brij52; TPS, Brij52; OPS, Brij52; DDPS, BrijC10; HPS, Brij30; OPS, Brij30. In some embodiments, the permeation enhancer may include at least two surfactants, e.g., TPS, BrijC10; TPS, Brij52; HPS, Brij52; HPS, BrijC10; DDPS, BrijC10; TPS, Brij30; or DDPS, Brij58. In some embodiments, the permeation enhance may include one of surfactant pairs DPS, Brij30 or TPS, BrijC10. In some embodiments, the permeation enhancer may exclude surfactant pair DPS, Brij30. In some embodiments, the permeation enhancer may exclude surfactant pair TPS, BrijC10.

A physical permeation enhancer may include an abrasive material, in solid or particulate form, for example, natural or synthetic woven or nonwoven abrasive fabrics or fibers/bristles; abrasive crystals of, e.g., quartz, metal, silica, alumina, silicon carbide, diamond, and derivatives thereof; natural or synthetic sponge; natural and synthetic abrasive particles commonly used in cosmetics, such as ground nut shells, ground seed pits, ground sea shells, diatoms, pumice or other minerals; polymer microbead; and the like. The physical permeation enhancer may also include, for example, microneedles, such as in a microneedle patch. The physical permeation enhancer may be used in combination with application of energy, for example, mechanical energy may be used with the abrasive material in a scrubbing, abrading, rubbing, or other motion to enhance permeation of the subject's integumentary system.

In various embodiments, the therapeutic agent may be provided in the form of a therapeutic composition. The therapeutic composition may be isotonic, e.g., including saline and/or a buffer effective to render the therapeutic composition isotonic for cells of any subject described herein, e.g., human cells. The therapeutic composition may be buffered, e.g., including an aqueous buffer such as phospho buffered saline, tris HCl buffered saline, borate buffered saline, HEPES buffered saline, and the like. The therapeutic composition may include the therapeutic agent in a percentage (w/v) of about one or more of: 0.00001, 0.0001, 0.001, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 12, 15, or 20, or a range between any two values thereof. The therapeutic composition may include the permeation enhancer in a percentage (w/v) of about one or more of: 0.00001, 0.0001, 0.001, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 12, 15, or 20, or a range between any two values thereof.

In some embodiments, the condition may include a pigmentation condition associated with the substrate including extracellular melanin. For example, the condition may include hyperpigmentation of the subject's skin associated with the substrate including extracellular melanin. The therapeutic agent may include an ionic photo-oxidant. The modulating may include driving the therapeutic agent into the subject's skin to contact the extracellular melanin. The method may include irradiating the therapeutic agent in the subject's skin effective to at least partly ameliorate the pigmentation condition in the subject's skin. For example, the method may reduce the appearance and/or persistence of the extracellular melanin in the subject's skin.

In various embodiments, the driving may be conducted using one or more of the applied energy, the chemical permeation enhancer, and the physical permeation enhancer. For example, the method may include applying electrical energy to iontophoretically drive the therapeutic agent into the subject's skin.

In several embodiments, the condition may include a pigmentation condition associated with the substrate including extracellular melanin. For example, the condition may include hyperpigmentation of the subject's skin associated with the substrate including extracellular melanin. The therapeutic agent may include an ionic melanin-binding agent. The modulating may include applying energy to drive, e.g., iontophoretically, the therapeutic agent into the subject's skin to contact the extracellular melanin. The therapeutic agent may contact the extracellular melanin effective to form a bound therapeutic agent:extracellular melanin complex. The method may also include extracting, e.g., iontophoretically, the bound therapeutic agent:extracellular melanin complex. The bound therapeutic agent:extracellular melanin complex may be extracted effective to at least partly ameliorate the pigmentation condition in the subject's skin. For example, the method may at least partly reduce the appearance and/or persistence of the extracellular melanin in the subject's skin.

In several embodiments, the extracting described herein may be one or more of active or passive. For example, the extracting may be actively conducted using one or more of the applied energy, the chemical permeation enhancer, and the physical permeation enhancer. For example, the method may include applying electrical energy to iontophoretically extract the ionic species, e.g., bound therapeutic agent:extracellular melanin complex, from the subject's skin, using, e.g., a reversed electrical field compared to that used to iontophoretically drive the therapeutic agent into the subject's skin. The extracting may include passive extraction, for example, allowing a period of time effective to passively extract a portion of the ionic species from the subject's integumentary system by one or more of exfoliation, metabolism, excretion by the subject, optionally with the passive assistance of one or more of the permeation enhancers described herein.

In various embodiments, the condition may include a pigmentation condition associated with the substrate including extracellular melanin. For example, the condition may include hyperpigmentation of the subject's skin associated with the substrate including extracellular melanin. The therapeutic agent may include an ionic photo-oxidant and melanin-binding agent. The modulating may include applying energy to drive, e.g., iontophoretically, the therapeutic agent into the subject's skin to contact the extracellular melanin. The therapeutic agent may contact the extracellular melanin effective to form a bound therapeutic agent:extracellular melanin complex. The method may also include irradiating the therapeutic agent in the subject's skin effective to react the extracellular melanin with the therapeutic agent to produce the reaction product, e.g., via photo-oxidation. The modulating may also include extracting, e.g., iontophoretically, from the subject's skin one or more of: an unbound portion of the therapeutic agent; the bound therapeutic agent:extracellular melanin complex; and the reaction product. The method may be effective to at least partly ameliorate the pigmentation condition in the subject's skin. For example, the method may at least partly reduce the appearance and/or persistence of the extracellular melanin in the subject's skin.

In several embodiments, the condition may include a pigmentation condition associated with the substrate comprising extracellular melanin. The therapeutic agent may include an ionic photo-oxidant and melanin-binding agent. The modulating may include applying energy to drive, e.g., iontophoretically, the therapeutic agent into the subject's skin to contact the extracellular melanin effective to form a bound therapeutic agent:extracellular melanin complex from a portion of the therapeutic agent. The method may further include extracting, e.g., iontophoretically, from the subject's skin an unbound portion of the therapeutic agent. The method may include irradiating the bound therapeutic agent:extracellular melanin complex in the subject's skin effective to form the reaction product of one or both of the therapeutic agent and the substrate. The method may include further extracting, e.g., iontophoretically, from the subject's skin one or more of: an unbound portion of the therapeutic agent; the bound therapeutic agent:extracellular melanin complex; and the reaction product. Extracting, e.g., iontophoretically, from the subject's skin an unbound portion of the therapeutic agent prior to irradiating the bound therapeutic agent:extracellular melanin complex in the subject's skin may reduce collateral damage to the subject's skin associated with irradiation of the unbound portion of the therapeutic agent. The method may be effective to at least partly ameliorate the pigmentation condition in the subject's skin. For example, the method may at least partly reduce the appearance and/or persistence of the extracellular melanin in the subject's skin.

In some embodiments, the condition may include a pigmentation condition. As used herein, a pigmentation condition includes, for example, disorders of hyperpigmentation, hypopigmentation, and/or irregular pigmentation. A pigmentation condition may include endogenous and/or exogenous causes. For example: lentigo may be associated with genetics, age (“senile lentigines”), solar exposure (“solar lentigines”); ephelides (freckles) may be associated with genetics and may be triggered or exacerbated by solar exposure, and the like.

Pigmentation disorders may arise from photo-reactions associated with sun exposure. Pigmentation disorders may arise from photo-reactions associated with the use of systemic or topical medications or contact with plants or foods in conjunction with sun exposure. Sun exposure in combination with administration of a photosensitive substance may lead to an erythematous allergic reaction, including lymphocytes, eosinophils, and edema, which may lead to a bullous reaction on sun-exposed skin, and eventually, hyperkeratosis and melanocytic hyperplasia leading to hyperpigmentation. Various substances may cause photosensitivity leading to pigmentation disorders, for example: anthranilic acids, e.g., meclofenamic acid; antibiotics, e.g., ceftazidime, fluoroquinolones, griseofulvin, ketoconazole, nalidixic acid, sulfonamides, tetracyclines, and trimethoprim; nonsteroidal anti-inflammatory drugs, e.g., ibuprofen, carprofen, benoxaprofen, arylpropionic acid derivatives, ketoprofen, nabumetone, naproxen, and tiaprofenic acid; antineoplastic agents, e.g., dacarbazine, fluorouracil, methotrexate, and vinblastine; coal tar; diuretics, e.g., furosemide, hydrochlorothiazide, and bendroflumethiazide; porphyrins; psoralens; pyrazolidinediones, e.g., oxyphenbutazone and phenylbutazone; dyes, e.g., eosin, fluorescein, methylene blue, and rose bengal; retinoids, e.g., etretinate and isotretinoin; salicylic acids, e.g., aspirin and diflunisal; amiodarone; desipramine; diltiazem; fibric acid derivatives; imipramine; phenothiazines; quinidine; quinine; sulfite food derivatives; plants or plant-derived substances, e.g., lemons, limes, fig leaves or stems, celery, dill, parsnips, and carrot juice; and the like.

Pigmentation disorders may arise from various substances, such as from those substances in the preceding paragraph or others, even without sun exposure. Substances which may lead to pigmentation disorders without significant sun exposure may include, for example: amiodarone; amitriptyline; metals, e.g., arsenic, bismuth, iron, gold, mercury, silver, and platinum; bleomycin, busulfan, clofazimine, cyclophosphamide, daunorubicin, doxorubicin, minocycline, platin chemotherapeutics such as cisplatin, nitrogen mustard, phenothiazines, zidovudine, and the like.

Pigmentation disorders may arise from other conditions or disorders, for example: conditions of adrenal insufficiency, in which hormones that stimulate melanin synthesis, such as melanocyte-stimulating hormone (MSH), may be elevated, e.g., Addison's disease and Nelson's syndrome; conditions involving elevated adrenocorticotropic hormone (ACTH), e.g., Cushing's disease; hemochromatosis; hyperthyroidism, e.g., Grave's disease; café au lait macules that may be associated with neurofibromatosis; melanoma; seborrheic keratosis; actinic keratosis; hyperpigmentation associated with insulin resistance, e.g., acanthosis nigricans; pigmentation associated with pregnancy or other hormone changes, such as melasma; cholasma, linea nigra, and aromatase deficiency; diabetic dermopathy; infections, such as tinea versicolor; Peutz-Jeghers syndrome; smoker's melanosis; celiac disease; Cronkite-Canada syndrome; porphyria; post-inflammatory hyperpigmentation; genetic disease; metabolic disease; cancer; renal disease; liver disease; autoimmune disease; and the like. It is to be understood that the preceding pigmentation disorders are examples and are not intended to be limiting.

In various embodiments, the subject may include one of: a human, a canine, a feline, an ungulate, a rodent, a reptile, or an avian. The subject's integumentary system may include any body surface tissue or organ, for example, one or more of: a skin, a mucous membrane, a cornea, a sclera, a dermal gland, a follicle, a nail, a cuticle, a nail bed, a hoof, a horn, a scale, a tooth, e.g., an enamel, and the like. In some embodiments, the subject's integument may include, for example, one or more of: a skin, a mucous membrane, a dermal gland, a follicle, a nail, a cuticle, a nail bed, a hoof, a horn, and a scale. For example, the subject's integumentary system or integument may include the subject's skin.

In some embodiments, the substrate may include one or more of: an endogenous pigment, an exogenous pigment, a biomolecule, a integumentary structure associated with the pigment condition, an integumentary cell associated with the pigment condition, pigmented byproducts of blood or inflammation, and the like. For example, the substrate may include an endogenous pigment, melanin, e.g., extracellular melanin.

An endogenous pigment is a pigment created by the subject, e.g., melanins such as eumelanin, pheomelanin, and the like. An endogenous pigment may be created by the subject's body in reaction to an exogenous substance, e.g., various pigments created by the body in binding exogenous metals, especially heavy metals. An exogenous pigment may be acquired by the subject from the environment by ingestion, skin absorption, infection, entry through a wound, and the like, e.g., a tattoo pigment.

In several embodiments, the therapeutic agent may include one or more of: a dye, a skin lightening agent, an oxidant, an agent that blocks synthesis or maturation of melanin, and the like.

The therapeutic agent may include, for example, one or more of: an ionic 3,7-diaminophenothiazinium dye, such as methylene blue, new methylene blue, thionine, toluidine blue O, azure A, azure B, azure C, and the like; an ionic triarylmethane dye, e.g., a methyl violet dye, a fuchsine dye, a fuchsone/phenol dye, a malachite green dye, a victoria blue dye, and the like; percarbonate salts, e.g., sodium percarbonate, ammonium percarbonate, a tetraalkylammonium percarbonate, and the like; perborate salts, such as sodium perborate; dithionite salts, such as sodium dithionite; and the like. For example, the therapeutic agent may include one or more of: an ionic 3,7-diaminophenothiazinium dye; an ionic triarylmethane dye; a percarbonate salt; a perborate salt; and a dithionite salt. Percarbonate, perborate, and dithionite anions may form salts with any pharmaceutically acceptable cation, e.g., cations of lithium, sodium, potassium, cesium, calcium, magnesium, ammonium, alkyl (e.g., mono, di, tri, and tetra) alkyl ammonium, other pharmaceutically acceptable cations, and the like. Further, for example, the therapeutic agent may include one or more of: methylene blue, new methylene blue, thionine, toluidine blue O, azure A, azure B, azure C, a methyl violet dye, a fuchsine dye, a fuchsone/phenol dye, a malachite green dye, a victoria blue dye, sodium percarbonate, ammonium percarbonate, a tetraalkylammonium percarbonate, sodium perborate, and sodium dithionite.

In various embodiments, the at least one ionic species, e.g., the therapeutic agent, may include or be formed as a pharmaceutically acceptable salt, or may include a pharmaceutically acceptable counter-ion. For example, the therapeutic agent may be an ionic therapeutic agent including or formed as a pharmaceutically acceptable salt including a pharmaceutically acceptable counter-ion. Compounds, such as therapeutic agents, may possess one or more of acidic and basic functional groups. Acidic functional groups may be reacted with any of a number of organic or inorganic bases to form a pharmaceutically acceptable salt. Basic functional groups may be reacted with any of a number of organic or inorganic acids to form a pharmaceutically acceptable salt. Acids commonly employed to form acid addition salts from compounds with basic groups may include inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Examples of pharmaceutically acceptable anions of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

Base addition salts may include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, and the like.

The therapeutic agents may be combined with an acceptable pharmaceutical carrier. Suitable pharmaceutical carriers may contain inert ingredients which do not interact with the compound. Standard pharmaceutical formulation techniques can be employed, such as those described in Remington's Pharmaceutical Sciences, Mack Publishing Company, Easton, Pa. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like.

The therapeutic agent may include an agent that comprises, forms, or facilitates formation of one or more of: hydrogen peroxide, a lipid peroxide, an organic peroxide, singlet oxygen, superoxide, an organic radical, and hydroxyl radical. For example, the therapeutic may include an ionic photo-oxidant, e.g., photosensitizing redox cycling dyes such as phenothiazinium dyes.

For example, compounds containing the 3,7-diaminophenothiazinium redox pharmacophore may be two-electron redox systems with standard reduction potentials, e.g., +0.01 V for methylene blue, that may be compatible with non-enzymatic and enzyme-dependent cycling between the oxidized dye form and the colorless reduced leuco form under cellular redox conditions. Spontaneous autoxidation of the leuco form of these phenothiazinium redox cyclers under physiological conditions may regenerate the dye form. Importantly, electron transfer from the leuco form to molecular oxygen may induce the non-enzymatic formation of reactive oxygen species including H₂O₂, and spontaneous ROS formation by PRC redox cycling may be driven by biological reducing agents including glutathione and NAD(P)H. Suitable 3,7-diaminophenothiazinium compounds, may include, for example:

methylene blue, e.g., as the chloride salt of 3,7-bis(dimethylamino)-phenothiazinium:

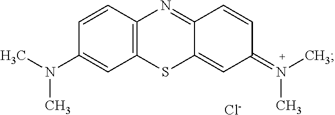

new methylene blue, e.g., as the chloride of 2,8-dimethyl-3,7-bis(ethylamino)-phenothiazinium:

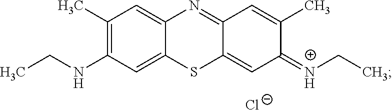 [SMILES: CCNc1cc2sc3c/c(=[NH+]/CC)c(C)cc-3nc2cc1C ; [Cl-]]

thionine, e.g., as the acetate salt of 3,7-diaminophenothiazinium:

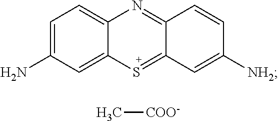

toluidine blue O, e.g., the chloride salt of 2-methyl-3-amino-7-dimethylaminophenothiazinium:

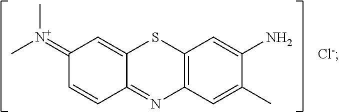

Azure A, e.g., as the chloride salt of 3-amino-7-dimethylamino-phenothiazinium:

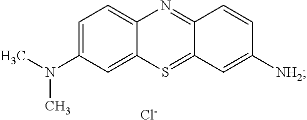

Azure B, e.g., as the chloride salt of 3-methylamino-7-dimethylamino-phenothiazinium:

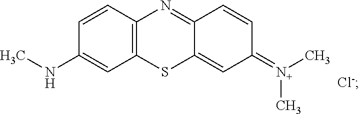

Azure C, e.g., as the chloride salt of 3-methylamino-7-amino-phenothiazinium:

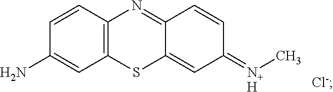

and the like. Each of the preceding dyes may be alternatively provided as a salt with any pharmaceutically acceptable anion. Moreover, each of the preceding dyes may be provided in an alternate redox state, e.g., the corresponding leuco forms.

An agent that blocks synthesis or maturation of melanin may include, for example, bone morphogenic protien-4 (BMP-4), an active fusion protein of BMP-4, an active fragment of BMP-4, a BMP-4 mimic or a combination thereof, as described in Yaar, et al., U.S. Pat. App. Pub. No. 20090053707, the entire contents of which are incorporated herein by reference.

In some embodiments, the substrate may include extracellular melanin. The method may be effective to at least partly reduce the appearance and/or persistence of the extracellular melanin in the subject's integumentary system, e.g., skin.

In several embodiments, the method may include reacting the therapeutic agent and the substrate effective to form the reaction product thereof in the subject's integumentary system. For example, the method may include irradiating the subject effective to cause a photochemical reaction. The photochemical reaction may include one or more of the substrate and the therapeutic agent. The photochemical reaction may produce the reaction product. The method may include irradiating the subject in a wavelength range that overlaps an absorption wavelength of the ionic species, e.g., the therapeutic agent, the bound therapeutic agent:substrate complex, and the like. The method may include irradiating the subject at a wavelength of one or more of: 400 nanometers (nm) to about 700 nm, between about 550 nm and about 700 nm, about 610 nm, and about 670 nm.

In various embodiments, the at least one ionic species may include the therapeutic agent. The modulating may include applying energy to drive, e.g., iontophoretically, the therapeutic agent into the subject's integumentary system effective to contact the substrate. The modulating may include extracting, e.g., iontophoretically, a portion of the at least one ionic species from the subject. The method may include extracting, e.g., iontophoretically, an unbound portion of the therapeutic agent from the subject's integumentary system. The modulating comprising modulating the therapeutic agent in the subject's integumentary system effective to at least partly avoid systemic administration of the therapeutic agent to the subject.

In some embodiments, the at least one ionic species may include the bound therapeutic agent:substrate complex. The modulating may include extracting, e.g., iontophoretically, a portion of the bound therapeutic agent:substrate complex from the subject's integumentary system. The at least one ionic species may include the reaction product. The modulating may include extracting, e.g., iontophoretically, a portion of the reaction product from the subject's integumentary system.

In several embodiments, the method may include allowing a period of time effective to passively extract a portion of at least one waste product from the subject, e.g., a period of time effective to allow one or more of exfoliation/epidermal maturation, metabolism, excretion, diffusion, and the like to remove a portion of at least one waste product from the subject. The period of time may be a time in days of one or more of about: 0.1, 0.5, 1, 1.5, 2, 2.5, 3, 3.5, 4, 4.5, 5, 5.5, 6, 6.5, 7, 7.5, 8, 9, 10, 11, 12, 13, 14, 15, 16, 17, 18, 19, 20, 21, 28, 35, 42, 49, 56, 63, 70, 77, 84, 91, and 98, or a range between any two of the preceding values, for example, between about 1 and about 21, between about 1 and about 14, between 2 and about 8, between about 4 and about 7, and the like. The at least one waste product may include one or more of: the therapeutic agent; the bound therapeutic agent:substrate complex; and the reaction product.

In some embodiments, the at least one ionic species may include one or more of: methylene blue; a binding product of the methylene blue and a melanin substrate; and a photochemical reaction product of one or more of the methylene blue and the melanin substrate.

In several embodiments, the method may include providing the subject in need of therapy for the condition including at least one lentigines lesion in the skin of the subject. The method may include driving methylene blue into the skin effective to contact melanin associated with the at least one lentigines lesion in the skin. The driving may be via iontophoresis. The method may include forming the reaction product of the methylene blue and the melanin at the at least one lentigines lesion in the skin. For example, the method may include allowing the methylene blue to bind to the melanin. The method may include irradiating the methylene blue:melanin complex at a wavelength effective to cause further reaction between the methylene blue and the melanin, for example, by photochemically generating a reactive oxygen species. The method may include passively or actively extracting one or more of the methylene blue, the melanin, and the reaction product from the skin effective to at least partly ameliorate the at least one lentigines lesion in the skin of the subject. For example, the method may include passively extracting via exfoliation/epidermal maturation, metabolism, excretion, and/or diffusion from the subject. The method may include actively extracting via iontophoresis.

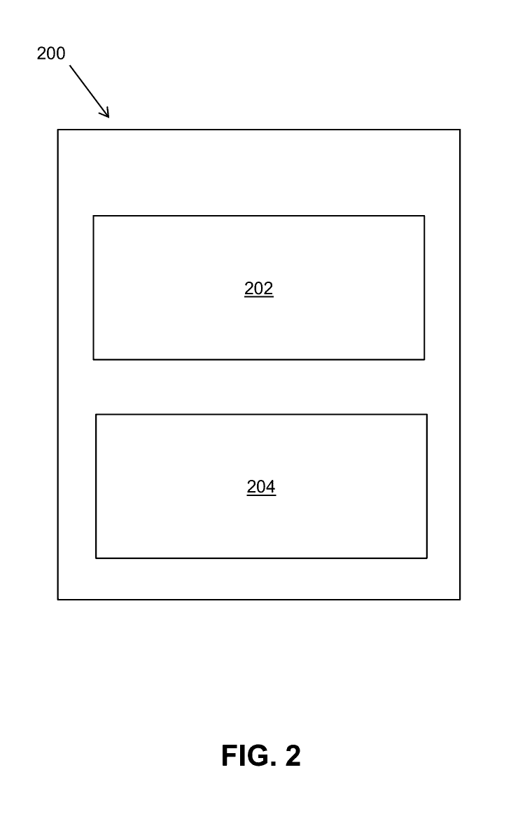

FIG. 2 is a block diagram illustrating an example kit for therapy 200. In various embodiments, kit 200 may include a therapeutic agent 202. Kit 200 may also include instructions 204. Instructions 204 may include directions to a user to perform any aspect of the method as described herein. For example, instructions 204 may direct a user to at least partly ameliorate a condition associated with a substrate located in a subject's integumentary system. The instructions to the user may include providing the subject in need of therapy for the condition. The instructions to the user may include contacting the therapeutic agent and the substrate in the subject's integumentary system. The instructions to the user may include modulating a depth of at least one ionic species in the subject's integumentary system. The at least one ionic species may include one or more of: the therapeutic agent; a bound therapeutic agent:substrate complex; and a reaction product of one or both of the therapeutic agent and the substrate.

Operation of the kit according to the instructions may be effective to at least partly ameliorate the condition associated with the substrate located in the subject's integumentary system, e.g., a pigmentation condition in the subject's skin. For example, the kit may at least partly reduce the appearance and/or persistence of extracellular melanin in the subject's skin.

The therapeutic agent in kit 200 may be contained within a pad or device (not shown) used for iontophoretic modulation. The therapeutic agent in kit 200 may be contained within a reservoir or container (not shown), and the kit may further include instructions directing a user to contact the therapeutic agent to the subject's integumentary system and/or the pad or device used for iontophoretic modulation. The therapeutic agent in kit 200 may also contain one or more permeation enhancers, buffers or other ionic components to help facilitate iontophoretic modulation.

In some embodiments of the kit, the therapeutic agent may include an ionic photo-oxidant. The instructions may include directing the user to modulate the depth of the at least one ionic species in the subject's integumentary system by applying energy to drive, e.g., iontophoretically, the therapeutic agent into the subject's skin effective to contact the substrate. The substrate may include extracellular melanin. The instructions may include directing the user to irradiate the therapeutic agent in the subject's skin effective to at least partly ameliorate the pigmentation condition in the subject's skin. For example, the irradiation may at least partly reduce the appearance and/or persistence of the extracellular melanin in the subject's skin.

In several embodiments of the kit, the therapeutic agent may include an ionic melanin-binding agent. The instructions may include directing the user to modulate the depth of the at least one ionic species in the subject's integumentary system by applying energy to drive, e.g., iontophoretically, the therapeutic agent into the subject's skin to contact the substrate. The therapeutic agent may contact the substrate effective to form a bound therapeutic agent:extracellular melanin complex. The substrate may include extracellular melanin. The instructions may include directing the user to extract, e.g., iontophoretically, the bound therapeutic agent:extracellular melanin complex effective to at least partly reduce the appearance and/or persistence of the extracellular melanin in the subject's integumentary system.

In various embodiments, the therapeutic agent may include an ionic photo-oxidant and melanin-binding agent. The instructions may include directing the user to modulate the depth of the at least one ionic species in the subject's integumentary system by applying energy to drive, e.g., iontophoretically, the therapeutic agent into the subject's skin to contact the substrate. The therapeutic agent may contact the substrate effective to form a bound therapeutic agent:extracellular melanin complex. The substrate may include extracellular melanin. The instructions may include directing the user to irradiate the therapeutic agent in the subject's skin effective to photochemically form the reaction product from one or both of the therapeutic agent and the substrate. The instructions may include directing the user to extract, e.g., iontophoretically, from the subject's skin one or more of: an unbound portion of the therapeutic agent; the bound therapeutic agent:extracellular melanin complex; and the reaction product.

In some embodiments, the instructions may describe the condition including a pigmentation condition. For example, the instructions may describe the condition including one or more of: hyperpigmentation, hypopigmentation, and irregular pigmentation. The instructions may describe the condition including one or more of: lentigines and ephelides.

In various embodiments, the instructions may describe the subject including one or more of: a human, a canine, a feline, an ungulate, a rodent, a reptile, or an avian. The instructions may describe the subject's integumentary system including any body surface tissue or organ, for example, one or more of: a skin, a mucous membrane, a cornea, a sclera, a dermal gland, a follicle, a nail, a cuticle, a nail bed, a hoof, a horn, a scale, and a tooth, e.g., an enamel. In some embodiments, the instructions may describe the subject's integument including, for example, one or more of: a skin, a mucous membrane, a dermal gland, a follicle, a nail, a cuticle, a nail bed, a hoof, a horn, and a scale. For example, the subject's integumentary system or integument may include the subject's skin.

In some embodiments, the instructions may describe the substrate including one or more of: an endogenous pigment, an exogenous pigment, a biomolecule, a integumentary structure associated with the pigment condition, an integumentary cell associated with the pigment condition, pigmented byproducts of blood or inflammation, and the like. For example, the instructions may describe the substrate including an endogenous pigment, melanin, e.g., extracellular melanin.

In various embodiments, the instructions may describe an endogenous pigment as a pigment created by the subject, e.g., melanins such as eumelanin, pheomelanin, and the like. An endogenous pigment may be created by the subject's body in reaction to an exogenous substance, e.g., various pigments created by the body in binding exogenous metals, especially heavy metals. The instructions may describe an exogenous pigment as acquired by the subject from the environment by ingestion, skin absorption, infection, entry through a wound, and the like, e.g., a tattoo pigment.

In some embodiments of the kit, the therapeutic agent may include one or more of: a dye, a skin lightening agent, an oxidant, an agent that blocks synthesis or maturation of melanin, and the like.

An agent that blocks synthesis or maturation of melanin may include, for example, bone morphogenic protien-4 (BMP-4), an active fusion protein of BMP-4, an active fragment of BMP-4, a BMP-4 mimic or a combination thereof.

The therapeutic agent may include, for example, one or more of: a 3,7-diaminophenothiazinium dye, such as methylene blue, new methylene blue, thionine, toluidine blue O, azure A, azure B, azure C, and the like; triarylmethane dyes, e.g., methyl violet dyes, fuchsine dyes, fuchsone/phenol dyes, malachite green dyes, and victoria blue dyes, and the like; percarbonate salts, e.g., sodium percarbonate, ammonium percarbonate, a tetraalkylammonium percarbonate, and the like; perborate salts, such as sodium perborate; dithionite salts, such as sodium dithionite; and the like. For example, the therapeutic agent may include one or more of: a 3,7-diaminophenothiazinium dye; a triarylmethane dye; a percarbonate salt; a perborate salt; and a dithionite salt. Percarbonate, perborate, and dithionite anions may form salts with any pharmaceutically acceptable cation, e.g., cations of lithium, sodium, potassium, cesium, calcium, magnesium, ammonium, alkyl (e.g., mono, di, tri, and tetra) alkyl ammonium, other pharmaceutically acceptable cations, and the like. Further, for example, the therapeutic agent may include one or more of: methylene blue, new methylene blue, thionine, toluidine blue O, azure A, azure B, azure C, a methyl violet dye, a fuchsine dye, a fuchsone/phenol dye, a malachite green dye, a victoria blue dye, sodium percarbonate, ammonium percarbonate, a tetraalkylammonium percarbonate, sodium perborate, and sodium dithionite.

The therapeutic agent may include an agent that comprises, forms, or facilitates formation of one or more of: hydrogen peroxide, a lipid peroxide, an organic peroxide, singlet oxygen, superoxide, an organic radical, and hydroxyl radical. For example, the therapeutic may include an ionic photo-oxidant, e.g., photosensitizing redox cycling dyes such as phenothiazinium dyes.

For example, compounds containing the 3,7-diaminophenothiazinium redox pharmacophore may be two-electron redox systems with standard reduction potentials, e.g., +0.01 V for methylene blue, that may be compatible with non-enzymatic and enzyme-dependent cycling between the oxidized dye form and the colorless reduced leuco form under cellular redox conditions. Spontaneous autoxidation of the leuco form of these phenothiazinium redox cyclers under physiological conditions may regenerate the dye form. Importantly, electron transfer from the leuco form to molecular oxygen may induce the non-enzymatic formation of reactive oxygen species including H₂O₂, and spontaneous ROS formation by PRC redox cycling may be driven by biological reducing agents including glutathione and NAD(P)H. Suitable 3,7-diaminophenothiazinium compounds, may include, for example: methylene blue, new methylene blue, thionine, toluidine blue O, Azure A, Azure B, Azure C, and the like. Each of the preceding dyes may be alternatively provided as a salt with any pharmaceutically acceptable anion. Moreover, each of the preceding dyes may be provided in an alternate redox state, e.g., the corresponding leuco forms.

In some embodiments, the therapeutic agent may be loaded in one or more of: an iontophoretic electrode; an electrode pad; an iontophoretic electrolyte vehicle, e.g., a conductive gel to be applied between the subject and an iontophoretic electrode; a reservoir; and the like. The iontophoretic electrode and/or electrode pad may be, for example, disposable, washable, and the like.

In several embodiments, the kit may include an iontophoresis apparatus. The instructions may direct the user to modulate the depth of the at least one ionic species in the subject's integumentary system using the iontophoresis apparatus. The iontophoresis apparatus may contain the therapeutic agent embedded into the apparatus, iontophoretic electrodes, or electrode pads containing the therapeutic agent in a form that can be, for example, immediately used. For example, the iontophoresis apparatus may include a set of self-contained, self-powered electrodes (such as that sold under the name IONTOPATCH 80™, SammonsPreston, obtained from Amazon.com, Seattle, Wash.) that may include or be combined with the therapeutic agent. The therapeutic agent may be mixed with one or more solvents, permeation enhancers, buffers or other ionic species that may facilitate modulation, operation, or performance of the iontophoresis apparatus.

In various embodiments, the instructions may include describing the condition including a pigmentation condition associated with the substrate including extracellular melanin. For example, the condition may include hyperpigmentation of the subject's skin associated with the substrate including extracellular melanin. The kit may be effective to at least partly ameliorate the pigmentation condition, e.g., to reduce the appearance and/or persistence of the extracellular melanin in the subject's integumentary system.

In some embodiments, the instructions may include directing the user to cause a reaction between the therapeutic agent and the substrate effective to form the reaction product thereof in the subject's integumentary system. For example, the instructions may include irradiating the subject effective to cause a photochemical reaction including one or more of the substrate and the therapeutic agent to produce the reaction product. The instructions may include irradiating the subject in a wavelength range that overlaps an absorption wavelength of the ionic species, e.g., the therapeutic agent, the bound therapeutic agent:substrate complex, and the like. The instructions may include irradiating the subject at a wavelength of one or more of: 400 nanometers (nm) to about 700 nm, between about 550 nm and about 700 nm, about 610 nm, and about 670 nm.

In several embodiments, the at least one ionic species may include the therapeutic agent. The instructions may include directing the user to modulate the depth of the at least one ionic species in the subject's integumentary system by applying energy to drive, e.g., iontophoretically, the therapeutic agent into the subject's integumentary system effective to contact the substrate. The instructions may include directing the user to modulate the depth of the at least one ionic species in the subject's integumentary system by extracting, e.g., iontophoretically, a portion of the at least one ionic species from the subject. The instructions may include directing the user to extract, e.g., iontophoretically, an unbound portion of the therapeutic agent from the subject's integumentary system.

In various embodiments, the at least one ionic species may include the bound therapeutic agent:substrate complex. The instructions may include directing the user to extract, e.g., iontophoretically, a portion of the bound therapeutic agent:substrate complex from the subject's integumentary system. The at least one ionic species may include the reaction product, and the instructions may include directing the user to extract, e.g., iontophoretically, a portion of the reaction product from the subject's integumentary system. The at least one ionic species may include the reaction product, and the instructions may further include directing the user to modulate the therapeutic agent in the subject's integumentary system effective to at least partly avoid systemic administration of the therapeutic agent to the subject.

In some embodiments, the instructions may include directing the user to allow a period of time effective to passively extract a portion of at least one waste product from the subject, e.g., a period of time effective to allow one or more of exfoliation/epidermal maturation, metabolism, excretion, diffusion, and the like to remove a portion of at least one waste product from the subject. The period of time may be a time in days of one or more of about: 0.1, 0.5, 1, 1.5, 2, 2.5, 3, 3.5, 4, 4.5, 5, 5.5, 6, 6.5, 7, 7.5, 8, 9, 10, 11, 12, 13, 14, 15, 16, 17, 18, 19, 20, 21, 28, 35, 42, 49, 56, 63, 70, 77, 84, 91, and 98, or a range between any two of the preceding values, for example, between about 1 and about 21, between about 1 and about 14, between 2 and about 8, between about 4 and about 7, and the like. The at least one waste product may include one or more of: the therapeutic agent; the bound therapeutic agent:substrate complex; and the reaction product. The at least one ionic species may include one or more of: methylene blue; a binding product of the methylene blue and a melanin substrate; and a photochemical reaction product of one or more of the methylene blue and the melanin substrate. The instructions may direct the user to passively extract the portion of the at least one waste product from the subject by one or more of exfoliation, metabolism, excretion, and diffusion.

In various embodiments, the instructions may direct the user to apply energy to the subject's integumentary system, the energy being effective to modulate the depth of the at least one ionic species with respect to the subject's integumentary system or to facilitate permeation of the at least one ionic species with respect to the subject's integumentary system. The energy may be any energy described herein, for example, one or more of mechanical energy, thermal energy, and electromagnetic energy. He instructions may direct the user to contact the subject's integumentary system with one or more of a chemical permeation enhancer and a physical permeation enhancer, e.g., any chemical permeation enhancer or physical permeation enhancer described herein. For example, the chemical permeation enhancer may include one or more of: a sulfoxide, an amide, a pyrrolidone, an alcohol, a glycol, an ester, a urea, a lactam, an enzyme, an imino sulfurane, a cyclodextrin, a fatty acid, an alkyl N,N di-substituted amino acetate, an essential oil, a polymer, and a surfactant. The physical permeation enhancer may include an abrasive or a plurality of microneedles. The kit may further include the therapeutic agent in a therapeutic composition. The therapeutic composition may be one or more of isotonic and buffered as described herein.

In several embodiments, the instructions in the kit may direct the user to conduct any aspect of the method described herein. The kit may include any therapeutic agent, mixture, permeation enhancer, or other component described herein, e.g. in the methods, compositions, and apparatus described herein.

In some embodiments, the kit may be in the form of a self-contained iontophoresis patch including a pair of iontophoretic electrodes coupled to a power supply. A cathodic electrode of the iontophoretic patch may be loaded with methylene blue and a chemical permeation enhancer together in a conductive solution, e.g., a conductive gel or an isotonic buffered saline solution. An anodic electrode of the iontophoretic patch may be loaded with a conductive gel, saline, etc, e.g., an isotonic buffered saline solution. The instructions may include providing the subject in need of therapy for the condition including at least one lentigines lesion in the skin of the subject. The instructions may include applying the pair of iontophoretic electrodes to the subject effective to place the cathodic electrode at the at least one lentigines lesion in the skin. The instructions may include allowing the self-contained iontophoresis patch to drive the methylene blue into the skin effective to contact melanin associated with the at least one lentigines lesion in the skin. The instructions may include forming the reaction product of the methylene blue and the melanin at the at least one lentigines lesion in the skin. For example, the instructions may include allowing the methylene blue to bind to the melanin. The instructions may include irradiating the methylene blue:melanin complex at a wavelength effective to cause further reaction between the methylene blue and the melanin, for example, by photochemically generating a reactive oxygen species. The irradiation instructions may include irradiation with sunlight. The kit may include an illuminating device, such as a light emitting diode device, and the instructions may include irradiation with the light emitting diode device. The instructions may include passively or actively extracting one or more of the methylene blue, the melanin, and the reaction product from the skin effective to at least partly ameliorate the at least one lentigines lesion in the skin of the subject. For example, the instructions may include directing the user to remove the patch and allow passive extraction to occur via exfoliation/epidermal maturation, metabolism, excretion, and/or diffusion from the subject. The kit may include a second iontophoresis patch loaded with buffered saline, and the instructions may direct the user to apply the second patch effective to conduct active extraction via iontophoresis.

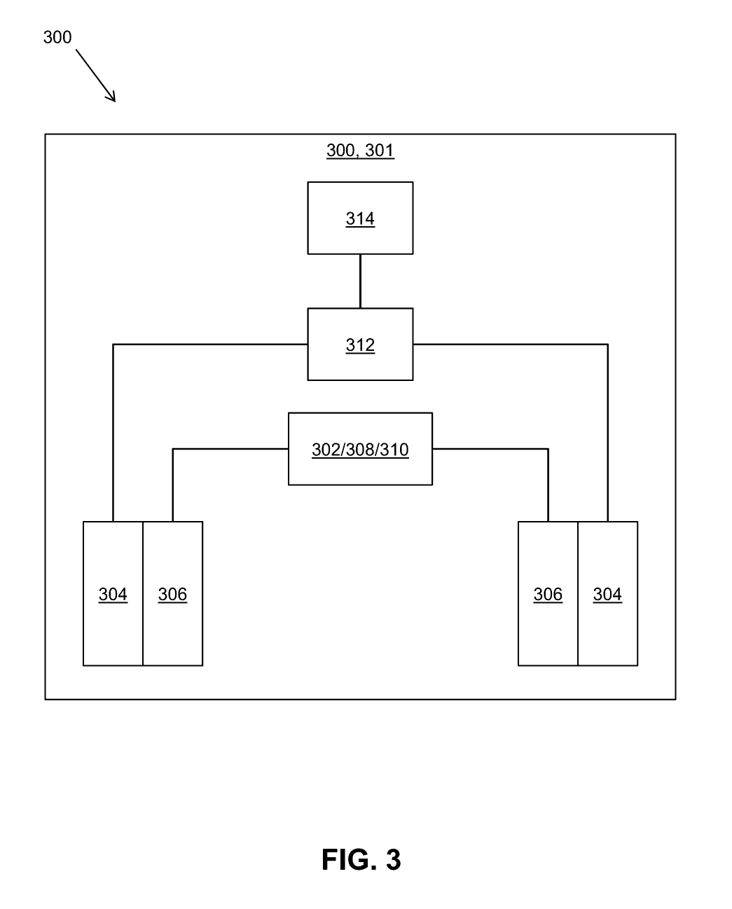

In various embodiments, an apparatus for therapy of a subject's integumentary system is provided. FIG. 3 is a block diagram of an exemplary iontophoresis apparatus 300. Apparatus 300 may include a therapeutic composition comprising a therapeutic agent 302. Therapeutic agent 302 may be any therapeutic agent described herein. For example, therapeutic agent 302 may include one or more of: a dye, a skin lightening agent, an oxidant, a reductant, and an agent that blocks synthesis or maturation of melanin. Apparatus 300 may include a mobilization module 301 configured to operatively couple one or more of energy or a permeation enhancer to the subject's integumentary system effective to modulate a depth of at least one ionic species in the subject's integumentary system or to modulate the permeation, e.g., mobility of the at least one ionic species in the subject's integumentary system. The subject's integumentary system may include a substrate associated with a condition in need of therapy. The at least one ionic species may include one or more of: the therapeutic agent; a bound therapeutic agent:substrate complex; and a reaction product of one or both of the therapeutic agent and the substrate.

In some embodiments, apparatus 300 may include therapeutic agent 302 loaded or impregnated in one or more of: one or more iontophoretic electrodes 304; one or more electrode pads 306; an iontophoretic electrolyte vehicle 308; and a reservoir 310. In FIG. 3, as an example, therapeutic agent 302 is depicted as loaded into reservoir 310 along with iontophoretic electrolyte vehicle 308. Mobilization module 301 may include one or more of: one or more iontophoretic electrodes 304; one or more electrode pads 306; reservoir 310; an iontophoretic circuit 312; and an iontophoretic power supply 314.

In several embodiments, therapeutic agent 302 may include one or more of: an ionic photo-oxidant, an ionic melanin-binding agent, and an ionic photo-oxidant and melanin-binding agent. Therapeutic agent 302 may include one or more of: a 3,7-diaminophenothiazinium dye; a triarylmethane dye; a percarbonate salt; a perborate salt; and a dithionite salt. Therapeutic agent 302 may include one or more of: methylene blue, new methylene blue, thionine, toluidine blue O, azure A, azure B, azure C, a methyl violet dye, a fuchsine dye, a fuchsone/phenol dye, a malachite green dye, a victoria blue dye, sodium percarbonate, ammonium percarbonate, a tetraalkylammonium percarbonate, sodium perborate, and sodium dithionite. Therapeutic agent 302 may include an agent that comprises, forms, or facilitates formation of one or more of: hydrogen peroxide, a lipid peroxide, an organic peroxide, singlet oxygen, superoxide, an organic radical, and hydroxyl radical.

In some embodiments, the therapeutic composition may include the therapeutic agent together with one or more of a chemical permeation enhancer, a physical permeation enhancer, an isotonic solution, and a buffered solution, e.g., as described herein. For example, the chemical permeation enhancer may include one or more of: a sulfoxide, an amide, a pyrrolidone, an alcohol, a glycol, an ester, a urea, a lactam, an enzyme, an imino sulfurane, a cyclodextrin, a fatty acid, an alkyl N,N di-substituted amino acetate, an essential oil, a polymer, and a surfactant. The physical permeation enhancer may include, for example, an abrasive. The apparatus may be configured for disposable, single-use application, e.g., as a patch. For example, the apparatus may be a self-contained self-powered iontophoretic patch preloaded with the therapeutic agent, and optionally the permeation enhancer, the isotonic solution, the buffer solution, and the like as described herein.

In some embodiments, the apparatus may be configured as an iontophoresis apparatus. For example, the apparatus may be configured as a self-contained patch wherein the mobilization module comprises a pair of iontophoretic electrodes coupled to a power supply. A cathodic electrode of the iontophoretic patch may be loaded with the therapeutic composition including methylene blue and a chemical permeation enhancer together in a conductive solution, e.g., a conductive gel, an isotonic buffered saline solution, and the like. An anodic electrode of the iontophoretic patch may be loaded with the conductive solution, e.g., a conductive gel, an isotonic buffered saline solution, and the like.

In various embodiments, a therapeutic composition is provided. The therapeutic composition may include a therapeutic agent. The therapeutic agent may include an ionic one or more of: a dye, a skin lightening agent, an oxidant, a reductant, and an agent that blocks synthesis or maturation of melanin. The therapeutic composition may include a permeation enhancer, e.g., a chemical permeation enhancer and/or a physical permeation enhancer as described herein. The therapeutic agent and the permeation enhancer may be combined together in an isotonic solution.

In some embodiments, the therapeutic composition may include any aspect of the therapeutic agent as described herein. For example, the therapeutic agent may include one or more of an ionic photo-oxidant, an ionic melanin-binding agent, and an ionic photo-oxidant and melanin-binding agent. The therapeutic agent may include one or more of: a 3,7-diaminophenothiazinium dye; a triarylmethane dye; a percarbonate salt; a perborate salt; and a dithionite salt. The therapeutic agent may include one or more of: methylene blue, new methylene blue, thionine, toluidine blue O, azure A, azure B, azure C, a methyl violet dye, a fuchsine dye, a fuchsone/phenol dye, a malachite green dye, a victoria blue dye, sodium percarbonate, ammonium percarbonate, a tetraalkylammonium percarbonate, sodium perborate, and sodium dithionite. The therapeutic agent may include an agent that comprises, forms, or facilitates formation of one or more of: hydrogen peroxide, a lipid peroxide, an organic peroxide, singlet oxygen, superoxide, an organic radical, and hydroxyl radical.

In several embodiments, the therapeutic composition may include any aspect of the chemical permeation enhancer and/or the physical permeation enhancer described herein. For example, the permeation enhancer may include one or more of: a sulfoxide, an amide, a pyrrolidone, an alcohol, a glycol, an ester, a urea, a lactam, an enzyme, an imino sulfurane, a cyclodextrin, a fatty acid, an alkyl N,N di-substituted amino acetate, an essential oil, a polymer, and a surfactant. The permeation enhancer may include an abrasive.

In some embodiments, the therapeutic composition may include any aspect of the isotonic solution as described herein. For example, the isotonic solution may be one or more of a saline solution and a buffer solution. The therapeutic composition may be formulated as a conductive solution, e.g., a conductive gel, the isotonic buffered saline solution, and the like.

In some embodiments, the therapeutic composition may include the therapeutic agent including methylene blue. The permeation enhancer may include a chemical permeation enhancer. The isotonic solution may be an isotonic buffered saline solution.

### EXAMPLES

### Example 1

### Iontophoresis Drives Methylene Blue into the Skin

An aqueous solution of 1% methylene blue (SigmaAldrich, St. Louis, Mo.) was prepared, and used to soak an absorbent iontophoresis electrode of a coupled pair of self-driven iontophoresis electrodes, about 3 inches in diameter, designated the “drive” electrode (a cathodic electrode of a coupled pair of self-driven iontophoresis electrodes sold as IONTOPATCH 80, SammonsPreston, manufactured by TRAVANTI MEDICAL, St. Paul Minn.).

A corresponding control preparation was made of an aqueous solution of 1% of a red anionic acid food dye (“acid orange”, SigmaAldrich, St. Louis, Mo.) and was used to soak a corresponding absorbent counter electrode of the coupled pair of self-driven iontophoresis electrodes, also about 3 inches in diameter, as a control. (A cationic control dye may be employed in other experiments, such as acid blue or acid green).

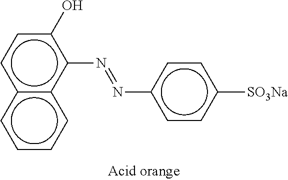

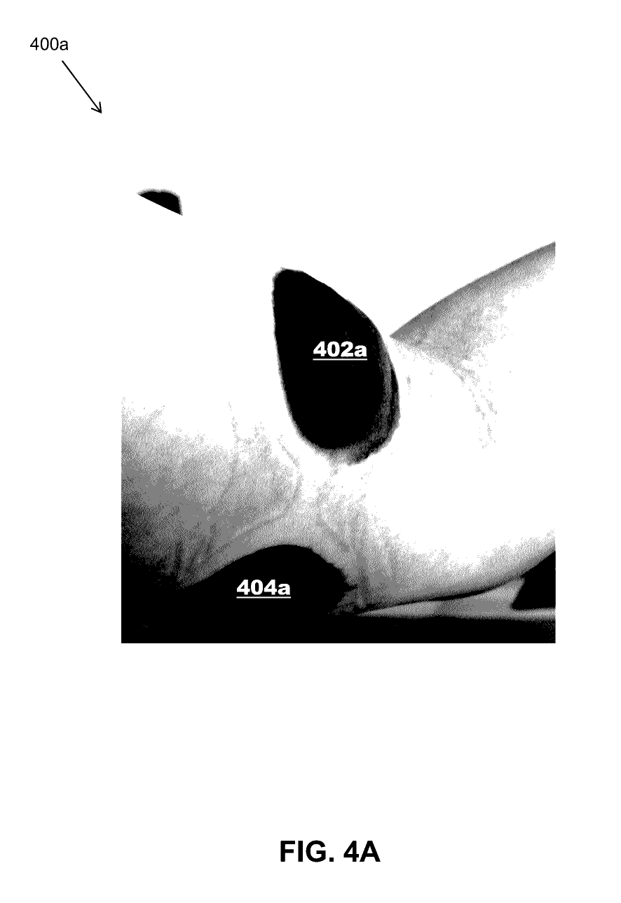

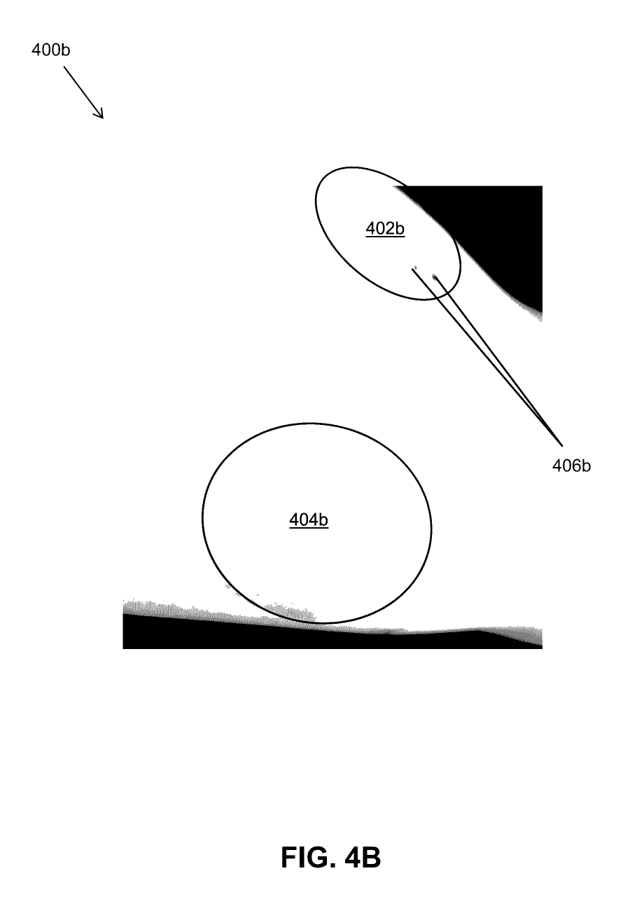

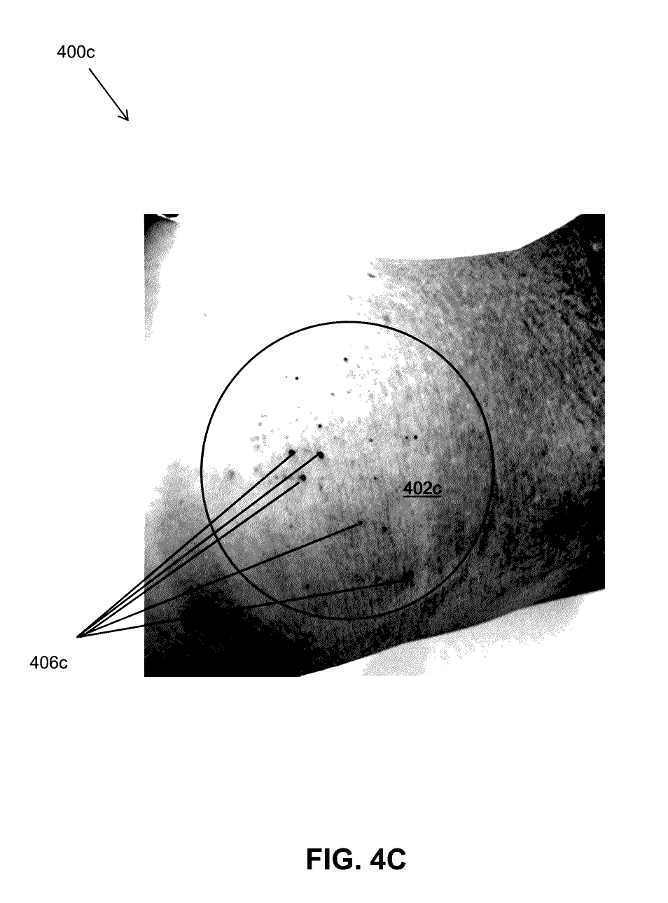

The drive electrode and the counter electrode were applied to the skin of a human volunteer on the inside of the subject's right upper arm, halfway between the shoulder and elbow, with about a 2 inch separation between electrode locations. The methylene blue drive electrode was located towards the subject's bicep and the acid orange control electrode was located towards the subject's triceps. The iontophoresis electrodes were operated according to manufacturer's instructions at a total current dosage of 80 mA-min over a period of about 14 hours (840 min), at which point the electrodes were removed from the subject, and the subject's skin was washed at the electrode locations. As depicted in image 400a in FIG. 4A, the subject's skin displayed a 3 inch diameter spot at the application location 402a of the drive electrode, where the subject's skin was markedly blue, and a 3 inch diameter spot at the application location 404a of the counter electrode, where the subject's skin was markedly red. Neither color could be washed off, indicating that the methylene blue and acid orange were incorporated at some depth in the subject's skin.

### Example 2

### Natural Exfoliation and Epidermal Maturation Remove Intradermal Dye from the Skin

Methylene blue and acid orange were driven into a subject's skin as described in EXAMPLE 1. The electrodes were removed from the subject and the subject's skin was washed at the electrode locations. No dye appeared to be located at the surface of the subject's skin, and neither the methylene blue nor the acid orange colors could be removed by vigorous washing.

The subject was examined about 4 days after removal of the electrodes. Both the methylene blue and acid orange appeared to be at least about 80% reduced in intensity over the 4-day period, indicating substantial epidermal maturation/exfoliation and removal of the intradermal dyes implanted in EXAMPLE 1. Image 400b in FIG. 4B shows the region of depleted blue color at the application location 402b of the drive electrode and a depleted red color at the application location 404b of the counter electrode. The subject was re-examined about 7 days after removal of the electrodes. Both the methylene blue and acid orange appeared to be at least about 90% reduced in intensity over the 7-day period, indicating substantial epidermal maturation/exfoliation and removal of the intradermal dyes implanted in EXAMPLE 1. Image 400c in FIG. 4C shows the region of depleted blue color at the application location 302b of the drive electrode and a depleted red color at the application location 404b of the counter electrode. The subject was re-examined about 7 days after removal of the electrodes.

At both 4 and 7 days, small, ˜0.1 mm spots of dye were observed, perhaps due to dye being retained in pores, hair follicles, or other dermal structures that may mature and exfoliate at a different effective rate than the skin overall.

### Prophetic Example 3

### Iontophoresis Removes Excess Intradermal Dye from the Skin

A skin location including a large, ˜0.5 mm diameter age spot may be selected in a human volunteer. The age spot may be characterized, e.g., for color and color density indicative of the amount of melanin present. Methylene blue may be driven into the subject's skin at the location of the age spot according to EXAMPLE 1, without using the acid orange control. The subject's skin at the location of the drive electrode may display a 3 inch solid blue circle as in EXAMPLE 1, with the age spot located within the circle. The subject's skin may be allowed to rest for about 5 min to permit the methylene blue to bind to extracellular melanin in the age spot. A set of the self-driven iontophoresis electrodes used in EXAMPLE 1, without any dye, may be applied with the locations of the electrodes swapped to place the control electrode above the methylene blue spot on the subject's skin. Swapping the electrodes may effectively reverse the iontophoresis polarity such that the control electrode acts to extract the methylene blue from the subject's skin. The self-driven iontophoresis electrodes may be operated to deliver a current dosage of 80 mA-min to the electrodes over a period of about 840 minutes as recommended by the manufacturer. The electrodes may be removed from the subject, and the subject's skin may be washed at the electrode locations. The subject's skin at the location of the drive electrode may be substantially depleted in the blue color. A portion of the blue color may be retained at the location of the age spot, indicating that the methylene blue may be bound to the melanin in the age spot.

### Prophetic Example 4

### Further Iontophoresis Removes a Portion of Dye-Bound Melanin from the Skin

The methylene blue retained at the location of the age spot and the melanin at the age spot may be characterized at the subject's skin at the end of EXAMPLE 3. A set of the self-driven iontophoresis electrodes used in EXAMPLE 1, without any dye, may be applied with the locations of the electrodes swapped to place the control electrode above the methylene blue spot on the subject's skin. The self-driven iontophoresis electrodes may be operated to deliver a current dosage of 80 mA-min to the electrodes over a period of about 840 minutes as recommended by the manufacturer. The electrodes may be removed from the subject and the subject's skin may be washed at the electrode locations. The subject's skin at the location of the age spot may be further depleted in one or more of the methylene blue color and the amount of melanin compared to the characterization at the beginning of this EXAMPLE, indicating that the reverse iontophoresis may remove a portion of bound methylene blue:melanin.

### Prophetic Example 5

### Irradiation Bleaches a Portion of Melanin in the Age Spot

The methylene blue retained at the location of the age spot and the melanin at the age spot may be characterized at the subject's skin as prepared at the end of EXAMPLE 3. The location of the age spot may be irradiated between about 400 nm to about 700 nm using a 100 W filtered xenon lamp for about 20 minutes. Subsequently, the location of the age spot may be characterized and the amount of visible methylene blue and melanin may be reduced compared to the beginning of this EXAMPLE.

### Prophetic Example 6

### Irradiation And Reverse Iontophoresis Removes Melanin from Age Spots

The methylene blue retained at the location of the age spot and the melanin at the age spot may be characterized at the subject's skin as prepared at the end of EXAMPLE 3. The location of the age spot may be irradiated between about 400 nm to about 700 nm using a 100 W filtered xenon lamp for about 20 minutes. A set of the self-driven iontophoresis electrodes used in EXAMPLE 1, without any dye, may be applied with the locations of the electrodes swapped to place the control electrode above the methylene blue spot on the subject's skin. The self-driven iontophoresis electrodes may be operated to deliver a current dosage of 80 mA-min to the electrodes over a period of about 840 minutes as recommended by the manufacturer. The electrodes may be removed from the subject and the subject's skin may be washed at the electrode locations. The subject's skin at the location of the age spot may be further depleted in one or more of the methylene blue color and the amount of melanin compared to the characterization at the beginning of this EXAMPLE, and that at the end of EXAMPLES 4 and 5.

### Example 7

### High Current Drives Methylene Blue into the Skin without Irritation

A pair of uncoupled adhesive electrophoresis electrodes, including a 1.5 mL capacity absorbent AgCl delivery electrode and a gel return electrode were obtained (Ionto+Plus Hi-Per Iontophoresis Electrode, Small, RICHMAR®, Chattanooga, Tenn.). An aqueous solution of 1% methylene blue was prepared in isotonic saline, of which 1.5 mL was added to the delivery electrode. A site was selected on a second human volunteer's upper thigh. The skin was shaved and cleaned with alcohol. The loaded delivery electrode was applied carefully according to the manufacturer's directions to ensure an adhesive seal, without leakage. The return electrode was also adhered about 4 inches from the delivery electrode. Both electrodes were located over muscle according to the manufacturer's directions. A commercial electrophoresis unit was coupled to the electrodes. Operating the delivery electrode as cathode and the return electrode as anode, current was applied at 1 mA for 80 min to result in a total current dosage of about 80 mA-min. Compared to the low current employed in Examples 1 and 2, the subject reported occasional tingling associated with the 1 mA current, but no discomfort. At about 60 min of dosage time, it was observed that the current occasionally tended to drop from 1 mA. Application of slight mechanical stimulation to the delivery electrode temporarily restored the current level to 1 mA, and was repeated as needed.

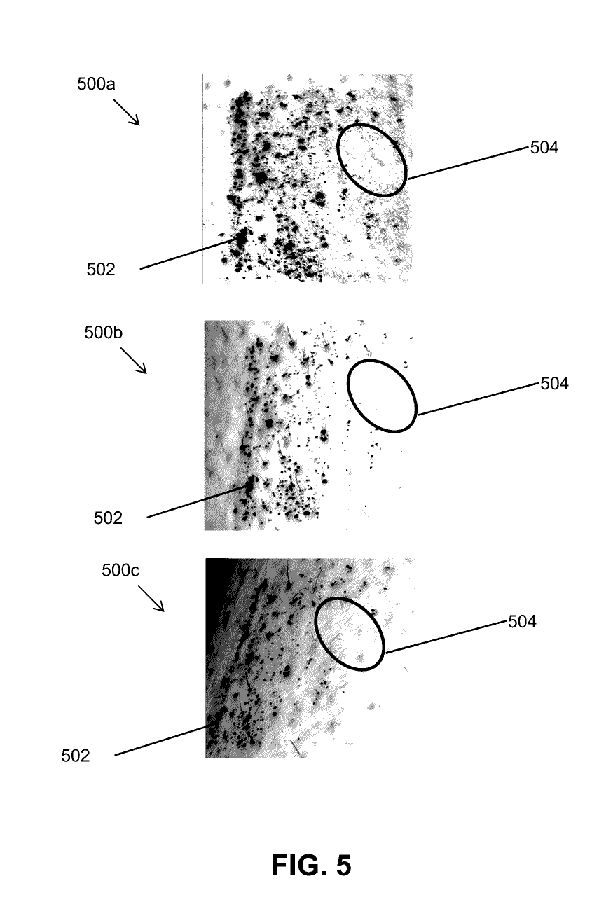

After 80 minutes, the current was shut off and the electrodes were removed from the subject. The subject's skin was washed at the location of the delivery electrode, where the subject's skin was markedly blue throughout. Darker blue localized spots, ˜0.1 mm-0.5 mm, were observed, which may correlate to skin pores or other breaks in the stratum corneum. The subject's skin was washed vigorously at the location of the delivery electrode, with scrubbing, which removed a significant amount of the dispersed dye, leaving mostly the darker blue localized spots 502, as depicted in image 500a in FIG. 5. The remaining dispersed dye 504 at the location of the delivery electrode had mostly disappeared with exfoliation over the course of 8 days, as depicted in image 500b in FIG. 5. The darker blue localized spots disappeared more slowly, with a significant amount still remaining at 12 days, as depicted in image 500c in FIG. 5. These results demonstrate that the methylene blue was delivered deep into the skin, because dispersed dye took about 8 days to disappear, and the localized dye persisted until at least 12 days. These results also suggest that skin pores or other breaks in the stratum corneum may be important for iontophoretic delivery of the methylene blue dye into the skin.

### Example 8

### High Current Drives Methylene Blue Into the Skin Without Irritation

It was observed that the pores in the skin of the second volunteer's skin at the upper thigh, as in Example 7, were large compared to the upper arm skin of the first volunteer as in Examples 1 and 2. Accordingly, a site was selected on the second human volunteer's upper arm, which was judged to have similar pore density to that in Examples 1 and 2. Electrodes were prepared, loaded, and applied to the second volunteer's upper arm as in Example 7. Current was applied at 1 mA for 80 min to result in a total current dosage of about 80 mA-min. Compared to Example 7, the subject reported somewhat increased tingling, but no discomfort, which may be associated with different skin sensitivity or thickness compared to the thigh location. Again, at about 60 min of dosage time, it was observed that the current occasionally tended to drop from 1 mA. Application of slight mechanical stimulation to the delivery electrode temporarily restored the current level to 1 mA, and was repeated as needed.

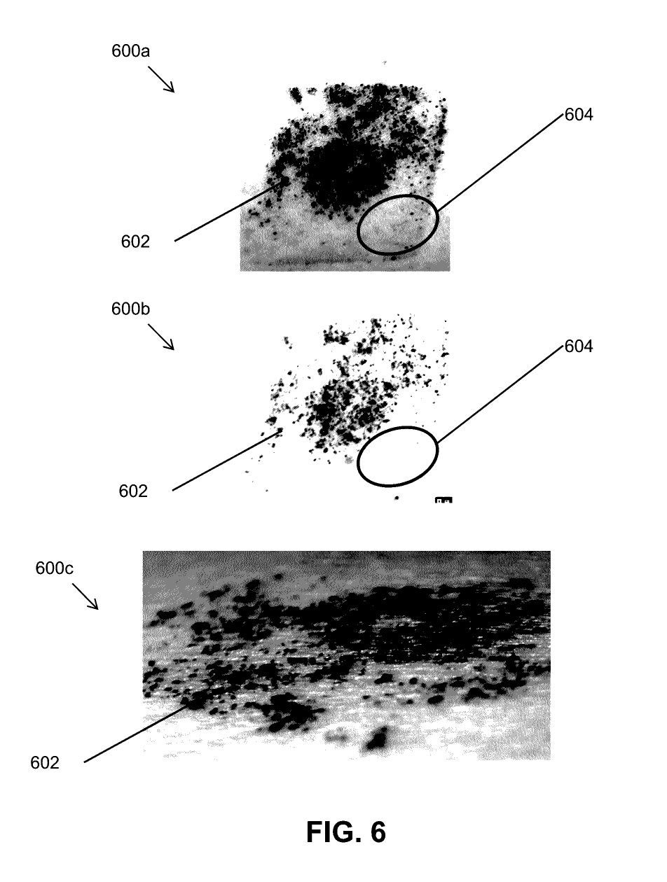

After 80 minutes, the current was shut off and the electrodes were removed from the subject. The subject's skin was washed at the location of the delivery electrode, where the subject's skin was markedly blue throughout. Darker blue localized spots, ˜0.1 mm-0.5 mm, were observed, which may correlate to skin pores or other breaks in the stratum corneum. The subject's skin was washed vigorously at the location of the delivery electrode, with scrubbing, which removed a significant amount of the dispersed dye, leaving mostly the darker blue localized spots 602, as depicted in image 600a in FIG. 6. It was noted that the localized dye spots could be felt as raised bumps. The remaining dispersed dye 604 at the location of the delivery electrode disappeared with exfoliation over the course of 7 days, as depicted in image 600b in FIG. 6. The darker blue localized spots 602 disappeared more slowly, with a significant amount still remaining at 12 days, as depicted in image 600c in FIG. 6. FIG. 6 is shot at an oblique angel to the skin and also shows that the raised bumps of localized dye 602 were still remaining at 12 days. These results demonstrate that the methylene blue was delivered deep into the skin, because dispersed dye took about 7 days to disappear, and the localized dye persisted until at least 12 days. These results also suggest that skin pores or other breaks in the stratum corneum may be important for iontophoretic delivery of the methylene blue dye into the skin. Further, the raised bumps of dye suggest that a limiting factor in dye penetration may be accumulation of dye, e.g., by clogging of pores or other breaks in the stratum corneum. The dye may precipitate or polymerize at such locations in the stratum corneum to form the raised bumps, may accumulate in pockets or voids at the skin, and the like.

### Example 9

### Iontophoresis Drives Methylene Blue Into the Skin

It was hypothesized that one or more surfactants might aid in dye penetration of the stratum corneum. Accordingly, as in Example 8, a site was selected on the second human volunteer's upper arm, electrodes were prepared, loaded, and attached, and current was applied at 1 mA for 80 min to result in a total current dosage of about 80 mA-min. In contrast to Example 8, the solution in the drive electrode was prepared as an isotonic saline solution containing 1% methylene blue and between about 1 to 5% surfactant including 3-(decyl dimethyl ammonio) propane sulfonate and polyoxyethylene (4) lauryl ether. The subject observed tingling but no discomfort. At about 70 min of dosage time, it was observed that the current occasionally tended to drop from 1 mA. Application of slight mechanical stimulation to the delivery electrode temporarily restored the current level to 1 mA, and was repeated as needed.

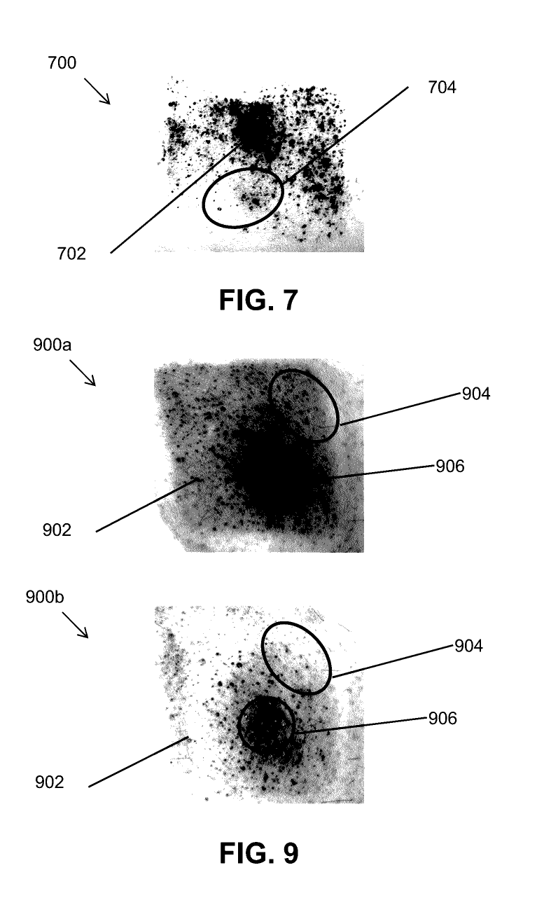

After 80 minutes, the current was shut off and the electrodes were removed from the subject. The subject's skin was washed at the location of the delivery electrode, where the subject's skin was markedly blue throughout. Darker blue localized spots, ˜0.1 mm-0.5 mm, were observed, which may correlate to skin pores or other breaks in the stratum corneum. The subject's skin was washed vigorously at the location of the delivery electrode, with scrubbing, which removed some of the dispersed dye, but still leaving a significant amount of dispersed dye. It was noted that the localized dye spots 702 again could be felt as raised bumps. The remaining dispersed dye 704 at the location of the delivery electrode was somewhat reduced, but did not disappear with exfoliation over the course of 7 days, as depicted in image 700 in FIG. 7.

These results demonstrate that the methylene blue was delivered deep into the skin using permeation enhancing surfactants, because both dispersed and localized dye persisted through at least 7 days. Moreover, these results show that permeation agents such as surfactants enhanced penetration of the dye into the stratum corneum generally, and not just at pores or other breaks in the stratum corneum. Also, because the current did not drop until 70 minutes, these results demonstrate that enhanced permeation of the dye into the stratum corneum generally, and not just at pores or other breaks in the stratum corneum, tends to mitigate clogging of pores or other breaks in the stratum corneum by the dye.

### Example 10

### Reverse Iontophoresis Extracts Methylene Blue Out of Skin

As in Example 9, a site was selected on the second human volunteer's upper arm, electrodes were prepared, loaded, and attached, and current was applied at 1 mA for 80 min to result in a total current dosage of about 80 mA-min. Also as in Example 9, the solution in the drive electrode was prepared as an isotonic saline solution containing 1% methylene blue and between about 1 to 5% surfactant including 3-(decyl dimethyl ammonio) propane sulfonate and polyoxyethylene (4) lauryl ether. The subject observed tingling but no discomfort. At about 70 min of dosage time, it was observed that the current occasionally tended to drop from 1 mA. Application of slight mechanical stimulation to the delivery electrode temporarily restored the current level to 1 mA, and was repeated as needed.

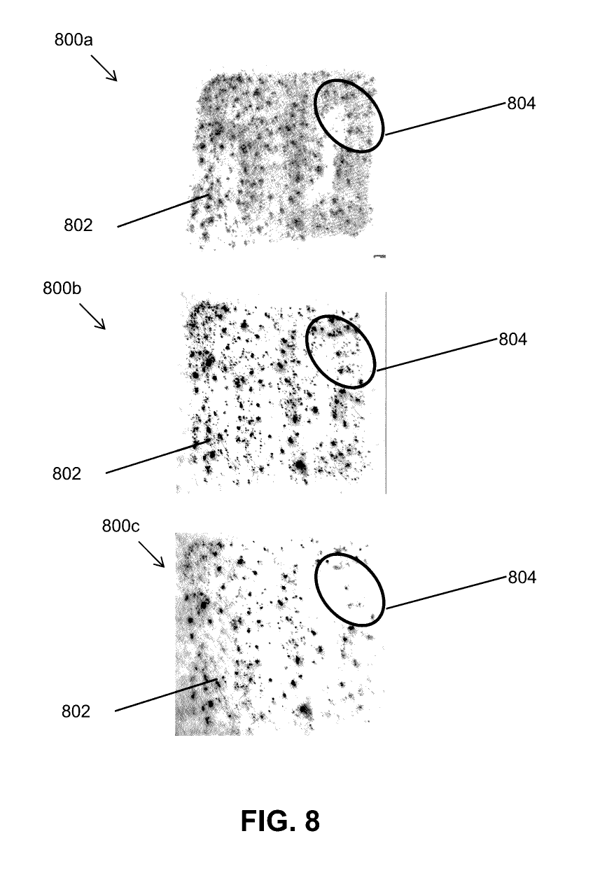

After 80 minutes, the current was shut off and the electrodes were removed from the subject. The subject's skin was washed at the location of the delivery electrode, where the subject's skin was markedly blue throughout. Darker blue localized spots, ˜0.1 mm-0.5 mm, were observed, which may correlate to skin pores or other breaks in the stratum corneum. The subject's skin was washed vigorously at the location of the delivery electrode, with scrubbing, which removed some of the dispersed dye, but still leaving a significant amount of dispersed dye, as depicted in image 800a in FIG. 8. It was noted that the localized dye spots again could be felt as raised bumps. The remaining dispersed dye at the location of the delivery electrode was somewhat reduced, but did not disappear with exfoliation over the course of 3 days.

At 6 days, a pair of delivery electrodes were prepared, each loaded with isotonic saline. The electrodes were applied at the original locations of the preceding electrodes in this example. However, the electrode leads were reversed, so that the electrode over the dye penetration region was operated as an anode. Current was applied at 1 mA for 80 min to result in a total current dosage of about 80 mA-min. No current drop was observed, in contrast to examples where the cathode was loaded with methylene blue dye. As shown by the before image 800b and after image 800c in FIG. 8, a significant amount of methylene blue dye was extracted from the subject's skin at the original dye penetration site. Additionally, it was observed that the extraction electrode was substantially blue. These results demonstrate that methylene blue dye may be extracted from the skin, even when located deep enough in the skin to withstand scrubbing and 6 days of exfoliation.

### Example 11

### Iontophoresis Drives Methylene Blue into the Skin

It was hypothesized that treating the skin with energy might aid in dye penetration of the stratum corneum, e.g. by ultrasonic energy delivered by sonication of the skin might aid in dye penetration of the stratum corneum. A site was prepared on the subject's upper arm near the elbow, and the elbow was partially submerged in water in a conventional 60 W/42 KHz jewelry sonicator, and sonicated for 20 s. The subject observed mild tingling but no discomfort.

As in Example 9, electrodes were prepared, loaded, and attached, with the drive electrode over the sonicated location and current was applied at 1 mA for 80 min to result in a total current dosage of about 80 mA-min. As in Example 9, the solution in the drive electrode was prepared as an isotonic saline solution containing 1% methylene blue and between about 1 to 5% surfactant including 3-(decyl dimethyl ammonio) propane sulfonate and polyoxyethylene (4) lauryl ether. The subject observed substantial prickly tingling, verging on discomfort. No current drop was observed.

After 80 minutes, the current was shut off and the electrodes were removed from the subject. The subject's skin was washed at the location of the delivery electrode, where the subject's skin was deeply blue throughout, much darker than the preceding Examples without sonication. Darker blue localized spots, ˜0.1 mm-0.5 mm, were observed, which may correlate to skin pores or other breaks in the stratum corneum. The subject's skin was washed vigorously at the location of the delivery electrode, with scrubbing, which left most of the localized and dispersed dye in the skin. It was noted that some lesser, but stable deposition occurred in areas not under the electrode, where excess MB/saline/surfactant solution contacted skin that had also been exposed to the sonicator.

The subject's skin was allowed to rest for 2 h, at which point a pair of delivery electrodes were prepared, each loaded with isotonic saline. The electrodes were applied at the original locations of the preceding electrodes in this Example. However, the electrode leads were reversed, so that the electrode over the dye penetration region was operated as an anode. Current was applied at 1 mA for 80 min to result in a total current dosage of about 80 mA-min. No current drop was observed. By visual examination, it was estimated that about 50% of methylene blue dye was extracted from the subject's skin at the original dye penetration site.

These results demonstrate that sonication, both alone and in combination with electrophoresis, assisted in delivery of the methylene blue deep into the skin. Moreover, sonication, along with the surfactants, increased permeation of the dye into the skin more generally and less localized to pores in the skin.

### Example 12

### Iontophoresis Drives Methylene Blue into the Skin

It was hypothesized that treating the skin with energy might aid in dye penetration of the stratum corneum, e.g. by mechanical energy delivered by rotary abrasion in combination with one or more surfactants.

A site was prepared on the subject's upper arm. A solution of the surfactant including 3-(decyl dimethyl ammonio) propane sulfonate and polyoxyethylene (4) lauryl ether was brushed into the skin using a soft bristled brush mechanically rotated at about 120 rpm for one minute. The subject observed no discomfort or irritation. The skin at the brushing site appeared soft, hydrated, and healthy.

A as in Example 11, the electrodes were prepared, loaded, and attached, and current was applied at 1 mA for 80 min to result in a total current dosage of about 80 mA-min. The solution in the drive electrode was prepared as an isotonic saline solution containing 1% methylene blue and between about 1 to 5% surfactant including 3-(decyl dimethyl ammonio) propane sulfonate and polyoxyethylene (4) lauryl ether. The drive electrode was applied over the brushing site. The subject observed tingling but no discomfort. No current drop was observed.

After 80 minutes, the current was shut off and the electrodes were removed from the subject. The subject's skin was washed at the location of the delivery electrode, where the subject's skin was markedly blue throughout. Some solution leaked out around the electrode and penetrated skin which had been brushed with the surfactant solution, but was not subject to iontophoresis. Darker blue localized spots 902, ˜0.1 mm-0.5 mm, were observed, which may correlate to skin pores or other breaks in the stratum corneum. The subject's skin was washed vigorously at the location of the delivery electrode, with scrubbing, which removed some of the dispersed dye 904, but still leaving a significant amount of dispersed dye 904, as depicted in image 900a in FIG. 9. In particular, significant pore-localized and general deposition was observed at a region 906 corresponding to the center of the rotary brushing. Since the rotary brushing was applied with pressure, and the brush had somewhat longer bristles at the center of rotation, the center of the rotary brushing received more vigorous brushing than other areas.

The subject's skin was allowed to rest for 2 h, at which point a pair of delivery electrodes were prepared, each loaded with isotonic saline. The electrodes were applied at the original locations of the preceding electrodes in this Example. However, the electrode leads were reversed, so that the electrode over the dye penetration region was operated as an anode. Current was applied at 1 mA for 80 min to result in a total current dosage of about 80 mA-min. No current drop was observed, in contrast to examples where the cathode was loaded with methylene blue dye. A significant amount of methylene blue dye was extracted from the subject's skin at the original dye penetration site, as demonstrated by image 900b in FIG. 9.

These results demonstrate that mechanical energy, both alone and in combination with electrophoresis, assisted in delivery of the methylene blue deep into the skin. Moreover, mechanical energy, along with the surfactants, increased permeation of the dye into the skin more generally and less localized to pores in the skin.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See Bryan A. Garner, A Dictionary of Modern Legal Usage 624 (2d. Ed. 1995). Also, to the extent that the terms “in” or “into” are used in the specification or the claims, it is intended to additionally mean “on” or “onto.” To the extent that the term “selectively” is used in the specification or the claims, it is intended to refer to a condition of a component wherein a user of the apparatus may activate or deactivate the feature or function of the component as is necessary or desired in use of the apparatus. To the extent that the terms “operatively coupled” or “operatively connected” are used in the specification or the claims, it is intended to mean that the identified components are connected in a way to perform a designated function. To the extent that the term “substantially” is used in the specification or the claims, it is intended to mean that the identified components have the relation or qualities indicated with degree of error as would be acceptable in the subject industry.

As used in the specification and the claims, the singular forms “a,” “an,” and “the” include the plural unless the singular is expressly specified. For example, reference to “a compound” may include a mixture of two or more compounds, as well as a single compound.

As used herein, the term “about” in conjunction with a number is intended to include ±10% of the number. In other words, “about 10” may mean from 9 to 11.

As used herein, the terms “optional” and “optionally” mean that the subsequently described circumstance may or may not occur, so that the description includes instances where the circumstance occurs and instances where it does not.

As stated above, while the present application has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art, having the benefit of the present application. Therefore, the application, in its broader aspects, is not limited to the specific details, illustrative examples shown, or any apparatus referred to. Departures may be made from such details, examples, and apparatuses without departing from the spirit or scope of the general inventive concept.

The various aspects and embodiments disclosed herein are for purposes of illustration and are not intended to be limiting, with the true scope and spirit being indicated by the following claims.

1. A method for therapy, comprising:
providing a subject in need of therapy for a condition, the condition being associated with a substrate located in the subject's integumentary system;
contacting a therapeutic agent and the substrate in the subject's integumentary system; and
modulating a depth of at least one ionic species in the subject's integumentary system, the at least one ionic species comprising one or more of: the therapeutic agent; a bound therapeutic agent:substrate complex; and a reaction product of one or both of the therapeutic agent and the substrate,
the method being effective to at least partly ameliorate the condition in the subject.

2. The method of claim 1, further comprising applying energy to the subject's integumentary system, the energy being effective to modulate the depth of the at least one ionic species with respect to the subject's integumentary system or to facilitate permeation of the at least one ionic species with respect to the subject's integumentary system.

3. The method of claim 1, further comprising contacting the subject's integumentary system with one or more of a chemical permeation enhancer and a physical permeation enhancer.

4. The method of claim 3, the chemical permeation enhancer comprising one or more of: a sulfoxide, an amide, a pyrrolidone, an alcohol, a glycol, an ester, a urea, a lactam, an enzyme, an imino sulfurane, a cyclodextrin, a fatty acid, an alkyl N,N di-substituted amino acetate, an essential oil, a polymer, and a surfactant.

5. The method of claim 1, the subject's integumentary system comprising the subject's skin and the condition comprising one or more of: lentigines and ephelides.

6. The method of claim 1, the therapeutic agent comprising one or more of: a dye, a skin lightening agent, an oxidant, a reductant, an agent that blocks synthesis or maturation of melanin; an ionic 3,7-diaminophenothiazinium dye; an ionic triarylmethane dye; a percarbonate salt; a perborate salt; a dithionite salt; methylene blue, new methylene blue, thionine, toluidine blue O, azure A, azure B, azure C, a methyl violet dye, a fuchsine dye, a fuchsone/phenol dye, a malachite green dye, a victoria blue dye, sodium percarbonate, ammonium percarbonate, a tetraalkylammonium percarbonate, sodium perborate, and sodium dithionite.

7. The method of claim 1, the substrate comprising extracellular melanin, the method being effective to at least partly reduce the appearance and/or persistence of the extracellular melanin in the subject's integumentary system.

8. The method of claim 1, further comprising one or more of:
applying energy to drive the therapeutic agent into the subject's integumentary system effective to contact the substrate;
extracting a portion of the at least one ionic species from the subject;
reacting the therapeutic agent and the substrate effective to form the reaction product thereof in the subject's integumentary system; and
irradiating the subject effective to cause a photochemical reaction comprising one or more of the substrate and the therapeutic agent to produce the reaction product;

9. The method of claim 1, comprising:
providing the subject in need of therapy for a condition, the condition comprising at least one lentigines lesion in the skin;
driving methylene blue into the skin effective to contact melanin associated with the at least one lentigines lesion in the skin;
forming the reaction product of the methylene blue and the melanin at the at least one lentigines lesion in the skin; and
passively or actively extracting one or more of the methylene blue, the melanin, and the reaction product from the skin effective to at least partly ameliorate the at least one lentigines lesion in the skin of the subject.

10. An apparatus for therapy of a subject's integumentary system, comprising:
a therapeutic composition comprising a therapeutic agent, the therapeutic agent comprising one or more of: a dye, a skin lightening agent, an oxidant, a reductant, and an agent that blocks synthesis or maturation of melanin; and
a mobilization module configured to operatively couple one or more of energy or a permeation enhancer to the subject's integumentary system effective to modulate a depth of at least one ionic species in the subject's integumentary system or modulate the mobility of the at least one ionic species in the subject's integumentary system,
the subject's integumentary system comprising a substrate associated with a condition in need of therapy, the at least one ionic species comprising one or more of: the therapeutic agent;
a bound therapeutic agent:substrate complex; and a reaction product of one or both of the therapeutic agent and the substrate.

11. The apparatus of claim 10, the mobilization module comprising one or more of: one or more iontophoretic electrodes; one or more electrode pads; a reservoir; an iontophoretic circuit; and an iontophoretic power supply, and
the apparatus comprising the therapeutic composition loaded or impregnated in one or more of: the one or more iontophoretic electrodes; the one or more electrode pads; an iontophoretic electrolyte vehicle; and the reservoir

12. The apparatus of claim 10, the therapeutic agent comprising one or more of: a dye, a skin lightening agent, an oxidant, a reductant, an agent that blocks synthesis or maturation of melanin; an ionic 3,7-diaminophenothiazinium dye; an ionic triarylmethane dye; a percarbonate salt; a perborate salt; a dithionite salt; methylene blue, new methylene blue, thionine, toluidine blue O, azure A, azure B, azure C, a methyl violet dye, a fuchsine dye, a fuchsone/phenol dye, a malachite green dye, a victoria blue dye, sodium percarbonate, ammonium percarbonate, a tetraalkylammonium percarbonate, sodium perborate, and sodium dithionite.

13. The apparatus of claim 10, the therapeutic composition comprising the therapeutic agent together with one or more of a chemical permeation enhancer, a physical permeation enhancer, an isotonic solution, and a buffered solution.

14. The apparatus of claim 10, configured for disposable, single-use application.

15. The apparatus of claim 10, configured as a self-contained patch wherein the mobilization module comprises a pair of iontophoretic electrodes coupled to a power supply, a cathodic electrode of the iontophoretic patch being loaded with the therapeutic composition, the therapeutic composition comprising methylene blue and a chemical permeation enhancer together in an isotonic buffered saline solution.

16. A therapeutic composition, comprising:
a therapeutic agent, the therapeutic agent comprising an ionic one or more of: a dye, a skin lightening agent, an oxidant, a reductant, and an agent that blocks synthesis or maturation of melanin; and
a permeation enhancer,
the therapeutic agent and the permeation enhancer being combined together in an isotonic solution.

17. The therapeutic composition of claim 15, the therapeutic agent comprising one or more of an ionic photo-oxidant, an ionic melanin-binding agent, and an ionic photo-oxidant and melanin-binding agent.

18. The therapeutic composition of claim 15, the therapeutic agent comprising one or more of: a dye, a skin lightening agent, an oxidant, a reductant, an agent that blocks synthesis or maturation of melanin; an ionic 3,7-diaminophenothiazinium dye; an ionic triarylmethane dye; a percarbonate salt; a perborate salt; a dithionite salt; methylene blue, new methylene blue, thionine, toluidine blue O, azure A, azure B, azure C, a methyl violet dye, a fuchsine dye, a fuchsone/phenol dye, a malachite green dye, a victoria blue dye, sodium percarbonate, ammonium percarbonate, a tetraalkylammonium percarbonate, sodium perborate, and sodium dithionite.

19. The therapeutic composition of claim 15, the permeation enhancer comprising one or more of: a sulfoxide, an amide, a pyrrolidone, an alcohol, a glycol, an ester, a urea, a lactam, an enzyme, an imino sulfurane, a cyclodextrin, a fatty acid, an alkyl N,N di-substituted amino acetate, an essential oil, a polymer, a surfactant, and an abrasive.

20. The therapeutic composition of claim 15, the therapeutic agent comprising methylene blue, the permeation enhancer comprising a chemical permeation enhancer, and the isotonic solution being an isotonic buffered saline solution.
